(19) United States
(12) Patent Application Publication
(10) Pub. No.: US 2023/0122932 A1
(43) Pub. Date: Apr. 20, 2023

(54) NEW SEX PHEROMONE COMPONENTS FOR THE FALL ARMYWORM, SPODOPTERA FRUGIPERDA

(71) Applicant: North Carolina State University, Raleigh, NC (US)

(72) Inventors: Coby Schal, Cary, NC (US); Eduardo Hatano, Meyrin (CH); Mohamed Saveer Ahmed, Raleigh, NC (US)

(73) Assignee: North Carolina State University, Raleigh, NC (US)

(21) Appl. No.: 17/909,671

(22) PCT Filed: Mar. 8, 2021
(86) PCT No.: PCT/US2021/021324

§ 371 Date: Sep. 6, 2022

Related U.S. Application Data
(60) Provisional application No. 62/986,419, filed on Mar. 6, 2020.

(51) Int. Cl. A01N 63/14; A01M 1/02

(52) U.S. Cl. CPC A01N 63/14; A01M 1/02

(57) ABSTRACT

Compositions for attracting and/or trapping male agricultural pests, particularly Spodoptera species (e.g., fall armyworm) are described. The compositions comprise two active components, one comprising a C7-C11 aldehyde and one comprising a C12-C16 aldehyde, acetate ester or primary alcohol. Also described are devices for attracting and/or trapping the agricultural pests and methods of controlling the population of the agricultural pests.

### CROSS-REFERENCE TO RELATED PATENT APPLICATIONS

This application claims benefit of U.S. Provisional Application Ser. No. 62/986,419, filed Mar. 6, 2020, the disclosure of which is incorporated herein by reference in its entirety.

### TECHNICAL FIELD

The presently disclosed subject matter relates to compositions, devices, and methods for attracting male agricultural pests, particularly male Spodoptera species pests, such as the fall armyworm (Spodoptera frugiperda). The compositions include a first component comprising a C7-C11 aldehyde and a second component comprising a C12-C16 aldehyde, acetate ester, or primary alcohol.

### Abbreviations

%=percentage
° C.=degrees Celsius
μg=microgram
μL=microliter
Ald=aldehyde
cm=centimeter
EAD=electroantennogram detector
FAW=fall armyworm
GC=gas chromatography
GLM=generalized linear model
I.D.=inner diameter
m=meter
mg=milligram
min=minutes
mL=milliliter
mm=millimeter
MS=mass spectroscopy
ng=nanogram
OAc=acetate
O.D.=outer diameter
psi=pounds per square inch
PTFE=polytetrafluoroethylene
s=seconds
SE=standard error
wt=weight

### BACKGROUND

The Fall Armyworm (FAW), Spodoptera frugiperda, is a global pest that feeds on leaves, stems, flowers and fruits of more than 350 plant species. It causes significant damage to cultivated grasses such as maize, rice, sorghum, sugarcane, and wheat; various vegetable crops; and cotton. Endemic to South America, FAW invaded North America decades ago, and in the last 3-5 years has become established in most of Africa, Yemen, the Indian subcontinent, Bangladesh, Thailand, Myanmar, Sri Lanka and most recently in China and Australia, causing devastating crop losses. FAW can cause >70% loss of maize yield in Africa, resulting in millions of US$ in economic losses and major challenges to food security. International agencies (e.g., the Food and Agricultural Organization of the United Nations (FAO)) consider control of FAW an international priority, especially because in its more recent invasive habitat it can produce several generations in a single season, and will likely become endemic.

Pheromone lures are manufactured and distributed internationally by various companies. However, currently available lures for FAW have limited trapping success and can also attract non-target insects that look like FAW.

Accordingly, there is an ongoing need to provide additional compositions, devices and methods of attracting Spodoptera species pests, such as FAW, and for controlling the populations of such pests.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

In some embodiments, the presently disclosed subject matter provides a composition for attracting a male agricultural pest of a Spodoptera species, wherein said composition comprises: (a) a first active component comprising at least one C7-C11 aldehyde; and (b) a second active component comprising at least one C12-C16 aldehyde, acetate ester, or primary alcohol. In some embodiments, the agricultural pest is Spodoptera frugiperda.

In some embodiments, the first active component comprises or consists of nonanal. In some embodiments, the second active component comprises a C12 acetate ester and/or a C14 acetate ester and/or a C16 acetate ester, optionally wherein said C12 acetate ester and/or C14 acetate ester and/or C16 acetate ester comprises an alkene group. In some embodiments, the second active component comprises (Z)-9-tetradecenyl acetate (Z9-14:OAc), (Z)-7-dodecenyl acetate (Z7-12:OAc), (Z)-9-dodecenyl acetate ester (Z9-12:OAc), (Z)-11-hexadecenyl acetate (Z11-16:OAc) or a combination thereof; optionally wherein the second active component comprises at least two of Z9-14:OAc, Z7-12:OAc, Z9-12:OAc, and Z11-16:OAc; further optionally wherein the second active component further comprises one or more additional component selected from the group comprising (Z)-10-tetradecenyl acetate (Z10-14:OAc), tetradecyl acetate (14:OAc), (Z)-11-tetradecenyl acetate (Z11-14:OAc), (E)-7-dodecenyl acetate (E7-12:OAc), dodecyl acetate (12:OAc), (Z)-11-dodecenyl acetate (Z11-12:OAc), (Z)-9-tetradecenal (Z9-14:Ald), and (Z)-11-hexadecenal (Z11-16:Ald).

In some embodiments, one or both of the first and the second active component is formulated in a slow release formulation, optionally wherein said first active component is formulated in an oil. In some embodiments, the first active component comprises nonanal and the second active component comprises Z9-14:OAc; and wherein the nonanal is present at an amount ranging from about 0.10 weight % to about 50 weight % compared to the weight of the Z9-14:OAc. In some embodiments, the nonanal is present at about 1 weight % compared to the weight of the Z9-14:OAc.

In some embodiments, the first active component and the second active component are separately formulated. In some embodiments, the composition further comprises a pest killing agent, a slow-acting insecticide, or a biological agent, optionally wherein said biological agent is selected from a bacteria, a fungi, a virus, and a nematode, optionally wherein said pest killing agent is a fast-acting insecticide.

In some embodiments, the presently disclosed subject matter provides a method of attracting a male agricultural pest of a Spodoptera species, the method comprising providing one or more baits or lures, wherein said one or more baits or lures collectively comprise (a) a first active component comprising at least one C7-C11 aldehyde; and (b) a second active component comprising at least one C12-C16 aldehyde, acetate ester, or primary alcohol. In some embodiments, the agricultural pest is Spodoptera frugiperda.

In some embodiments, the first active component comprises or consists of nonanal. In some embodiments, the second active component comprises a C12 acetate ester and/or a C14 acetate ester and/or C16 acetate ester, optionally wherein said C12 acetate ester and/or said C14 acetate ester and/or said C16 acetate ester comprises an alkene group. In some embodiments, the second active component comprises Z9-14:OAc, Z7-12:OAc, Z9-12:OAc, Z11-16:OAc or a combination thereof; optionally wherein the second active component comprises at least two of Z9-14:OAc, Z7-12:OAc, Z9-12:OAc, and Z11-16:OAc; further optionally wherein the second active component further comprises one or more additional component selected from the group comprising Z10-14:OAc, 14:OAc, Z11-14:OAc, E7-12:OAc, 12:OAc, Z11-12:OAc, Z9-14:Ald, and Z11-16:Ald.

In some embodiments, one or both of the first and the second active component is formulated in a slow release formulation, optionally wherein the first component is formulated in an oil. In some embodiments, the first active component comprises nonanal and the second active component comprises Z9-14:OAc; and wherein the nonanal is present in an amount ranging from about 0.10 weight % to about 50 weight % compared to the weight of the Z9-14:OAc. In some embodiments, the nonanal is present at about 1 weight % compared to the weight of the Z9-14:OAc.

In some embodiments, the first active component and the second active component are separately formulated, optionally wherein the first active component is provided in a separate dispenser from the second active component. In some embodiments, the first active component is formulated in an oil and provided in a first dispenser and the second active component is provided in a second dispenser, optionally wherein said second dispenser comprises rubber.

In some embodiments, the one or more baits or lures are provided in association with a housing for trapping one or more pest and the method further comprises collecting one or more male agricultural pest of a Spodoptera species in the housing. In some embodiments, the method further comprises estimating a pest population size based upon analyzing the number of pests trapped in the housing. In some embodiments, the method further comprises keeping trapped male pests in said housing or transferring said trapped pests to another housing, thereby controlling a pest population by removing male pests from the total pest population and reducing the number of male pests available for mating.

In some embodiments, the method further comprises controlling a pest population by treating attracted, optionally trapped, male pests with a slow-acting insecticide or biological control agent; and releasing the treated male pests, wherein the treated male pests transfer the slow-acting insecticide or biological control agent to female pests upon mating. In some embodiments, the method further comprises controlling a pest population by providing a plurality of the one or more baits or lures to a select location, thereby inundating the location with the first and second components to confuse male agricultural pests and make it more difficult for said male agricultural pests to locate a mate. In some embodiments, the method further comprises controlling a pest population by treating the attracted, optionally trapped male pests with a pest killing agent, optionally a fast-acting insecticide. In some embodiments, the method is performed at or near a port of entrance, optionally at or near an imported container at a harbor, airport, or roadway and/or train border crossing, to detect the presence or absence of said pest.

In some embodiments, the presently disclosed subject matter provides a multi-component device for attracting and capturing a male agricultural pest of a Spodoptera species, the device comprising: (a) a first active component comprising at least one C7-C11 aldehyde; (b) a second active component comprising at least one C12-C16 aldehyde, acetate ester, or primary alcohol; (c) a housing comprising one or more opening for entry of said male agricultural pest, optionally wherein said housing further comprises a mount configured to mount the device in a fixed position; and (d) one or more dispensers, wherein first active component (a) is incorporated into at least one of said one or more dispensers and wherein second active component (b) is incorporated into at least one of said one or more dispensers. In some embodiments, at least one of said one or more dispensers is made of a chemically neutral material selected from the group comprising a polymer, a glass, a rubber, an elastomer, cellulose, wood, and felt. In some embodiments, the housing further comprises an insert comprising an adhesive that can adhere to said pest to keep said pest from exiting the housing. In some embodiments, the device further comprises (e) a further active agent comprising a killing agent, a slow acting insecticide, or a biological agent, optionally wherein said biological agent is selected from the group comprising a bacteria, a virus, a fungi, and a nematode.

Accordingly, it is an object of the presently disclosed subject matter to provide compositions for attracting male FAW (i.e., male agricultural pests of the species Spodoptera frugiperda) and/or males of other Spodoptera species, as well as to provide related methods and devices. This and other objects are achieved in whole or in part by the presently disclosed subject matter. Further, an object of the presently disclosed subject matter having been stated above, other objects and advantages of the presently disclosed subject matter will become apparent to those skilled in the art after a study of the following description, Figures, and Examples.

### BRIEF DESCRIPTION OF THE FIGURES

Reference will now be made to the accompanying drawings, which are not necessarily drawn to scale.

FIG. 1 is a series of chromatograms and graphs showing the antennal electrophysiological responses of male Spodoptera frugiperda to a female sex pheromone gland extract. (I) is a chromatogram of clean hexane and (II) is a graph of the respective electrophysiological response. (III) is a chromatogram of one female-equivalent gland extract and (IV) is a graph of the respective electrophysiological response. The nonanal peak and electrophysiological response are indicated by the dashed line in (III) and (IV). The main pheromone component, (Z)-9-tetradecenyl acetate (Z9-14:OAc), is also indicated. Antennal response is represented by the median of gas chromatography-electroantennogram detector (GC-EAD) recordings (N=3),

FIG. 2 is a series of graphs showing the behavioral responses (activation, close approach, and contact) of male Spodoptera frugiperda to synthetic pheromone formulations with different doses of nonanal in an olfactometer assay. Bars represent proportions of attracted moths (N=15) using, from left to right for each set of five bars, a control formulation (unfilled bar), a 2-component pheromone blend of (Z)-9-tetradecenyl acetate (Z9-14:OAc) and (Z)-7-dodecenyl acetate (Z7-12:OAc) that resembles commercial formulations (light grey bar), the 2-component pheromone blend further including 0.05% nonanal (medium grey bar), the 2-component pheromone blend further including 0.1% nonanal (dark grey bar), and the 2-component pheromone blend further including 1% nonanal (black bar). Pheromone-triggered response of males was enhanced by nonanal at different concentrations (0.05, 0.1 and 1% relative to 100 nanograms (ng) of Z9-14:OAc).

FIG. 3 is a graph of the number of male S. frugiperda caught per day in traps with a pheromone mix and nonanal in a cotton field. Nonanal was added relative to the amount of (Z)-9-tetradecenyl acetate (Z9-14:OAc (00 micrograms (μg))). Traps with a pheromone mix of Z9-14:OAc (100 μg), (Z)-7-dodecenyl acetate (Z7-12:OAc; 0.58 μg), and 1% nonanal (black) had the highest number of males caught, followed by traps with a pheromone mix with 0.1% added nonanal (gray), the pheromone mix alone (light gray) and the control. Three traps per treatment were examined over six days. Bars represent mean catches (±standard error (SE)). Different letters denote significantly different catches (Poisson GLM, p<0.05, N=6).

FIG. 4 is a graph of the number of male S. frugiperda caught per day in traps with nonanal, a 2-component pheromone mix, or a combination of nonanal and the 2-component pheromone mix in a cotton field. Nonanal was added relative to the amount of (Z)-9-tetradecenyl acetate (Z9-14:OAc (1 milligram (mg))). Traps with a pheromone mix and 1% nonanal had the highest number of males caught. The treatments with nonanal alone (1% and 4%) did not catch any males. Three traps per treatment were examined over nine days. Bars represent mean catches (±standard error (SE)). Different letters denote significantly different trap catches (Poisson GLM, p<0.05, N=7-9).

FIGS. 5A and 5B are a pair of graphs showing the number of male S. frugiperda caught per day in traps with commercial pheromone lures from commercial supplier A (Scentry Biologicals, Billings, Mont., United States of America) (FIG. 5A), and commercial supplier B (Trece Incorporated, Adair, Okla., United States of America) (FIG. 5B) with or without nonanal (20 micrograms (μg)) in a sorghum field. The addition of nonanal to pheromone lures (gray bars) significantly increased the number of males caught compared to the commercial lures alone, Four traps per treatment were examined over four days. Bars represent mean catches (±standard error (SE)). Different letters denote significantly different catches (Poisson GLM, p<0.05, N=4).

FIG. 6 is a graph showing the number of male S. frugiperda caught per day in traps with a commercial pheromone lure (Scentry Biologicals, Billings, Mont., United States of America) and nonanal in a sorghum field. Nonanal (20 micrograms (μg)) was tested in combination with the lure. The addition of nonanal to the pheromone lure (gray or black bars) significantly increased the number of males caught compared to the commercial lures alone. Four traps per treatment were examined along five days. Bars represent mean catches (±standard error (SE)). Different letters denote significantly different catches (Poisson GLM, p<0.05, N=5).

### DETAILED DESCRIPTION

FAW has been reported to damage field crops, including: alfalfa, barley, Bermuda grass, buckwheat, cotton, clover, corn, oat, millet, peanut, rice, ryegrass, sorghum, sugarbeet, sudangrass, soybean, sugarcane, timothy, tobacco, and wheat, sweet corn, apple, grape, orange, papaya, peach, strawberry and a number of flowers. Previously identified components of the FAW pheromone generally include 12-, 14- and 16-carbon acetate esters and aldehydes, such as (Z)-9-tetradecenyl acetate (Z9-14:OAc), (Z)-9-tetradecenal (Z9-14:Ald), tetradecyl acetate (14:OAc), (Z)-10-tetradecenyl acetate (Z10-14:OAc), (Z)-11-tetradecenal acetate (Z1-14:OAc), (Z)-7-dodecenyl acetate (Z7-12:OAc, (E)-7-dodecenyl acetate (E7-12:OAc), (Z)-9-dodecenyl acetate (Z9-12:OAc), dodecyl acetate (12:OAc), (Z)-11-dodecenyl acetate (Z11-12:OAc), (Z)-11-hexadecenyl acetate (Z11-16:OAc), and (Z)-11-hexadecenal (Z11-16:Ald). These and other known FAW pheromone components are described, for example, in The Pherobase: Database of Pheromones and Semiochemicals (which can be accessed online at pherobase.com). The corresponding primary alcohols can also be components of Spodoptera sex pheromones. For example, C12-C16 primary alcohols, e.g., monounsaturated C12-C16 primary alcohols, such as (Z)-7-dodecen-1-ol, (Z)-9-tetradecen-1-ol, and (Z)-11-hexadecen-1-ol, have been identified as pheromone components in some Spodoptera species. Without being bound to any one theory, these primary alcohols are likely biosynthetic precursors of acetate ester pheromone components. Thus, while to date they have not been identified as pheromone components of FAW, it is possible that they occur in the FAW pheromone gland.

Commercial FAW lure formulations typically include two of these components, Z9-14:OAc and Z7-12:OAc, and one or more of the other components, most commonly Z9-12:OAc and Z11-16:OAc. According to the presently disclosed subject matter, it has been found that female FAW also produce nonanal (9-carbon aldehyde; 9:Ald), and that adding nonanal to previously identified sex pheromone components can significantly increase trap catch. Currently, known pheromone components are formulated in commercial lures without nonanal. As described herein, the combination of nonanal with commercial lures and the combination of nonanal with mixtures of known pheromone components have been studied.

The presently disclosed subject matter will now be described more fully. The presently disclosed subject matter can, however, be embodied in different forms and should not be construed as limited to the embodiments set forth herein below and in the accompanying Examples. Rather, these embodiments are provided so that this disclosure will be thorough and complete, and will fully convey the scope of the embodiments to those skilled in the art.

All references listed herein, including but not limited to all patents, patent applications and publications thereof, and scientific journal articles, are incorporated herein by reference in their entireties to the extent that they supplement, explain, provide a background for, or teach methodology, techniques, and/or compositions employed herein.

### I. Definitions

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

Definitions of specific chemical functional groups and chemical terms are those that would be understood by one of ordinary skill in the art. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas N. Sorrell (2006) Organic Chemistry, 2^{nd} Edition, University Science Books, South Orange, N.J.; Smith & March (2001) March's Advanced Organic Chemistry, 5th Edition, John Wiley & Sons, Inc., New York; Larock (1989) Comprehensive Organic Transformations, VCH Publishers, Inc., New York; Carruthers (1987) Some Modern Methods of Organic Synthesis, 3^{rd} Edition, Cambridge University Press, Cambridge, 1987; the entire contents of each of which are incorporated herein by reference.

As used herein and in the appended claims, the singular forms “a,” “an,” and “the” include plural referents unless the context clearly dictates otherwise. For example, a pheromone component refers to one or more pheromone components. As such, the terms “a”, “an”, “one or more” and “at least one” can be used interchangeably.

The term “and/or” when used in describing two or more items or conditions, refers to situations where all named items or conditions are present or applicable, or to situations wherein only one (or less than all) of the items or conditions is present or applicable.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” can mean at least a second or more.

The term “comprising”, which is synonymous with “including,” “containing,” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

Unless otherwise indicated, all numbers expressing quantities of concentration, volume, weight, length, width, diameter, thickness, temperature, enzymatic activity, pH, time, mass ratio, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about”, when referring to a value is meant to encompass variations of in one example ±20% or ±10%, in another example ±5%, in another example ±1%, and in still another example ±0.1% from the specified amount, as such variations are appropriate to perform the disclosed methods.

Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4).

The terms “optional” and “optionally” as used herein indicate that the subsequently described event, circumstance, element, and/or method step may or may not occur and/or be present, and that the description includes instances where said event, circumstance, element, or method step occurs and/or is present as well as instances where it does not.

The terms “polymer” and “polymeric” refer to chemical structures that have repeating units (i.e., multiple copies of a given chemical substructure). As used herein, polymers can, in some embodiments, refer to structures having more than 3, 4, 5, 6, 7, 8, 9, or 10 repeating units and/or to structures wherein the repeating unit is other than methylene. Polymers can be formed from polymerizable monomers. A polymerizable monomer is a molecule that comprises one or more reactive moieties {e.g., siloxy ethers, hydroxyls, amines, vinylic groups (i.e., carbon-carbon double bonds), halides (i.e., Cl, Br, F, and I), esters, carboxylic acids, activated esters, and the like} that can react to form bonds with other molecules. Generally, each polymerizable monomer molecule can bond to two or more other molecules. In some cases, a polymerizable monomer will bond to only one other molecule, forming a terminus of the polymeric material. Some polymers contain biodegradable linkages, such as esters or amides, such that they can degrade overtime under biological conditions.

The terms “pheromone”, “sex pheromone”, “pheromone compound” “pheromone component” and the like as used herein can refer to a volatile, intraspecies specific signal molecule produced and released by an insect (e.g., a female insect) at the time of, or prior to, mating that attracts an opposite sex insect (e.g., a male insect).

In some embodiments, chemical compounds (e.g., pheromone compounds) are referred to herein by the number of carbon atoms present in the compound or in a main carbon chain of the compound. For example, C12-C16 aldehydes are compounds that contain a twelve, thirteen, fourteen, fifteen, or sixteen carbon atom chain where one of the carbon atoms in the carbon atom chain is the carbon atom of an aldehyde group (i.e., a group having the formula —C(═O)—H). In some embodiments, the carbon atom of the aldehyde group is at an end of the carbon atom chain. Similarly, C7-C11 aldehydes are compounds that contain a seven, eight, nine, ten, or eleven carbon atom chain, respectively, where one of the carbon atoms in the chain is the carbon atom of an aldehyde group.

C12-C16 acetates (or acetate esters) are compounds that comprise a carbon atom chain comprising twelve, thirteen, fourteen, fifteen or sixteen carbon atoms, where one of the carbon atoms is substituted by (i.e., covalently bonded to) an oxygen atom which is also attached to an acetyl group (i.e., a —C(═O)CH₃ group), thereby forming an ester of the formula RO—C(═O)CH₃, where R is the C12-C16 carbon atom chain. In some embodiments, the oxygen atom covalently bonded to the acetyl group is attached to a carbon atom at one end of the carbon atom chain. In some embodiments, the acetyl group can be replaced by a similar acyl group, e.g., —C(═O)—CH₂CH₃, —C(═O)CH(CH₃)₂, or —C(═O)CH₂CH₂CH₃.

C12-C16 primary alcohols are compounds that comprise a carbon atom chain comprising twelve, thirteen, fourteen, fifteen, or sixteen carbon atoms where one of the carbon atoms is the carbon atom of a primary alcohol group (i.e., a —CH₂OH group). Thus, C12-C16 primary alcohols include, but are not limited to, (Z)-7-dodecen-1-ol, (Z)-9-tetradecen-1-ol, and (Z)-11-hexadecen-1-ol.

The carbon atom chain of the aldehydes, acetate esters, or primary alcohols described herein can be fully saturated or have one or more sites of unsaturation (i.e., one or more alkene or alkyne groups). In some embodiments, the aldehydes, acetate esters, or primary alcohols described herein can include one or more (e.g., 1 or 2) alkene groups. The carbon atom chains of the acetate esters, aldehydes, and primary alcohols can be straight or branched. In some embodiments, the aldehydes and/or acetate esters and/or primary alcohols described herein are straight-chain compounds.

Pheromones described herein can be referred to using IUPAC nomenclature or various abbreviations and derivations. For example, (Z)-hexadec-11-en-1-al, can also be written as Z-11-hexadecen-1-al, Z-11-hexadecenal, or Z-x-y:Ald, wherein x represents the position of the double bond, and y represents the number of carbons in the hydrocarbon skeleton. Abbreviations used herein and known to those skilled in the art to identify functional groups on the hydrocarbon skeleton include “Ald,” indicating an aldehyde, “OH,” indicating an alcohol, and “Ac,” indicating an acetyl. Also, the number of carbons in the chain can be indicated using numerals rather than using the written name. Thus, as used herein, an unsaturated carbon chain comprised of sixteen carbons can be written as hexadecene or 16.

As used herein, the term “isomer” refers to a molecule having the same chemical formula as another molecule, but with a different chemical structure. That is, isomers contain the same number of atoms of each element but have different arrangements of their atoms. Isomers include “structural isomers” and “stereoisomers.” In “structural isomers” (also referred to as “constitutional isomers”), the atoms have a different bond-sequence. Structural isomers have different IUPAC names and can include skeletal isomers, where hydrocarbon chains have variable amounts of branching, and positional isomers, which deals with the position of a functional group on a chain; and functional group isomerism, in which the molecular formula is the same but the functional group is different. The term “positional isomer” refers to a first compound which has the same carbon skeleton and functional group as a second compound but differs in the location of the functional group on or in the carbon skeleton. In stereoisomers, the bond structure is the same, but the geometrical positioning of atoms and functional groups in space differs. This class of isomers includes enantiomers, which are isomers that are non-superimposable mirror-images of each other, and diastereomers, which are stereoisomers that are not mirror-images. Geometric isomers or cis/trans isomers are diastereomers with a different stereochemical orientation at a bond. E/Z isomers, which are a subset of geometric isomers, are isomers with a different geometric arrangement at a double bond. Another type of isomer, conformational isomers (conformers), may be rotamers, diastereomers, or enantiomers depending on the exact compound.

An “effective amount” means that amount of a composition or component thereof that is sufficient to affect desired results. An effective amount can be administered in one or more administrations. For example, an effective amount of the composition can refer to an amount of a pheromone composition that is sufficient to attract a given insect to a given location. In some embodiments, an effective amount of the composition can refer to an amount that is sufficient to disrupt mating of a particular insect population of interest in a given locality.

The terms “pest” and “agricultural pest” as used herein refer to any Spodoptera species, e.g., Spodoptera frugiperda, that causes damage to a plant species, typically an agricultural crop.

The term “pest control agent” as used herein refers to compounds, organisms, or other agents that can be used to control or help to control a pest population. Pest control agents include attractants, such as pheromones, as well as chemical and biological agents that can kill pests, such as chemical insecticides and insecticidal microorganisms, e.g., bacteria (e.g., Bacillus thuringiensis), viruses, fungi, etc. The term “killing agent” as used herein can refer to an agent (e.g., a chemical insecticide) that kills pests too rapidly for the pest to pass the agent on to another pest. Thus, killing agents include fast-acting insecticides, chemical agents that can kill insects within minutes or hours. However, killing agents are not limited to toxic chemicals. They also include agents that can be used to drown, suffocate, or electrocute insects.

The term “insecticide” as used herein refers to any compound which kills insects or insect pests. In some embodiments, the term insecticide refers to a chemical agent that kills insects or is toxic to insects.

The term “active compound” as used herein refers to a sex pheromone for a Spodoptera species, optionally Spodoptera frugiperda. The active compound can be a compound produced by one gender of the Spodoptera species (e.g., in the female pheromone gland) or be or a compound structurally related thereto that can also attract individuals of one or more Spodoptera species.

The term “lure” refers to a composition comprising an active compound that acts as an attractant.

The term “bait” refers to a composition comprising an active compound that acts as an attractant in mixture with a feeding stimulant.

The term “adhesive” as used herein refers to a compound or material that is sticky and to which insects will adhere. Non-limiting examples of adhesives include, but are not limited to glue, starch, honey, pectin, gluten, an adhesive tape, etc.

### II. Compostions and Devices

The major constituents of the FAW sex pheromone were identified over three decades ago using gas chromatography-mass spectrometry (GC-MS) analysis of compounds from the female FAW pheromone gland. However, despite this, currently available lures for FAW have limited trapping success and can also attract non-target insects that look like FAW. According to one aspect, the presently disclosed subject matter relates to the discovery of new FAW pheromone components and their use to improve the performance of the current commercially available pheromone lures for FAW.

For example, the previously used GC-MS-based approaches for identifying sex pheromones in FAW often miss low-abundance compounds that can be highly attractive to the insect. According to the presently disclosed subject matter, a GC-electroantennogram detector (GC-EAD), a device that couples a GC to an insect antenna, the olfactory organ of insects that serves as a biological detector, was used to detect additional sex pheromone components. In this device, the GC separates chemicals in a mixture and presents them to the antenna, and its electrophysiological responses reveal which chemicals it senses. Through millions of years of evolution, the antenna has been tuned to species-specific pheromone components with sensitivity hundreds to thousands-fold greater than MS detectors. Briefly, as described further hereinbelow, an additional organic compound that occurs in tiny amounts in FAW females was identified. Although not previously identified as a FAW sex pheromone via GC-MS methods, this compound, i.e., nonanal (also known as nonanaldehyde, pelargonaldehyde, Aldehyde C9, or 9:Ald) gave robust antennal responses in the GC-EAD. Behavioral studies under laboratory and field conditions also indicated that this compound significantly improved attraction to commercial FAW lures.

Thus, in some embodiments, the presently disclosed subject matter provides a composition for attracting male FAW (i.e., male agricultural pests of the species Spodoptera frugiperda) and/or males of other Spodoptera species, wherein the composition comprises nonanal and/or a closely related compound (e.g., another C7-C11 aldehyde, such as heptanal, octanal, decanal or undecanal). The composition can further comprise one or more additional, previously identified sex pheromone components of a Spodoptera species, such as at least one or more saturated or unsaturated C12-C16 aldehyde, C12-C16 acetate ester, or C12-C16 primary alcohol (e.g., one or more saturated or unsaturated C12, C14, or C16 aldehyde, acetate ester, or primary alcohol). In some embodiments, the composition can further comprise one or more additional pest control agent, such as a fast- or slow-acting insecticide or a biological pest control agent.

In some embodiments, the presently disclosed subject matter provides a composition for attracting a male agricultural pest of a Spodoptera species, wherein said composition comprises: (a) a first active component comprising, consisting essentially of, or consisting of at least one C7-C11 aldehyde; and (b) a second active component comprising, consisting essentially of, or consisting of at least one C12-C16 aldehyde, acetate ester, or primary alcohol. In some embodiments, the male agricultural pest attracted by the composition comprises or consists of male FAW (Spodoptera frugiperda). Other Spodoptera species that can be attracted by the composition (either in addition to or instead of FAW) include, but are not limited to, S. cilium (grasslawn armyworm), S. dolichos (sweet potato armyworm), S. eridania (southern armyworm), S. exempta (African armyworm), S. exigua (beet armyworm), S. latifascia (velvet armyworm), S. littoralis (African cotton leafworm), S. litura (oriental leafworm moth) S. mauritia (lawn armyworm), S. pectinicornis (water-lettuce moth), S. praefica (western yellowstriped armyworm), and S. pulchella (Caribbean armyworm moth). In some embodiments, the composition attracts a single Spodoptera species. In some embodiments, the composition attracts more than one Spodoptera species.

In some embodiments, the first active component comprises, consists essentially of, or consists of nonanal (9:Ald). In some embodiments, the first active component comprises or consists of another C7-C11 aldehyde, such as, but not limited to, another straight chain C7-C11 aldehyde, i.e., heptanal, octanal, decanal, or undecanal. In some embodiments, the C7-C11 aldehyde can include one or more sites of unsaturation and/or can have a branched carbon atom chain Although to date these other C7-C11 aldehyde compounds have not been found in the FAW female's pheromone gland, it is possible that FAW pheromone receptors can be broadly tuned to nonanal-related aldehydes and/or that one or more of these other aldehydes will be detected in more concentrated FAW female pheromone gland extracts.

In some embodiments, the second active component comprises one or more of the C12-C16 aldehyde, acetate ester, or primary alcohol including, but not limited to, C12, C14, and C16 acetate esters and aldehydes. In some embodiments, the acetate ester or aldehyde is monounsaturated and the C12, C14 or C16 acetate ester or aldehyde comprises one alkene group. For example, in some embodiments, the acetate ester or aldehyde is selected from the group comprising (Z)-9-tetradecenyl acetate (Z9-14:OAc), (Z)-9-tetradecenal (Z9-14:Ald), (Z)-10-tetradecenyl acetate (Z10-14:OAc), (Z)-11-tetradecenal acetate (Z11-14:OAc), (Z)-7-dodecenyl acetate (Z7-12:OAc), (E)-7-dodecenyl acetate (E7-12:OAc), (Z)-9-dodecenyl acetate (Z9-12:OAc), (Z)-11-dodecenyl acetate (Z11-12:OAc), (Z)-11-hexadecenyl acetate (Z11-16:OAc), and (Z)-11-hexadecenal (Z11-16:Ald). In some embodiments, the second active component include a saturated C12-C16 aldehyde or acetate ester, such as tetradecyl acetate (14:OAc) or dodecyl acetate (12:OAc). In some embodiments, the second active component comprises one or more C12-C16 primary alcohol. In some embodiments, the primary alcohol is monounsaturated. In some embodiments, the primary alcohol is selected from (7)-7-dodecen-1-ol, (Z)-9-tetradecen-1-ol, and (Z)-11-hexadecen-1-ol. In some embodiments, the second active component comprises one, two, three, four, five, six or more compounds selected from the group comprising C12-16 acetate esters, C12-C16 aldehydes, and C12-C16 primary alcohols.

In some embodiments, the second active component comprises a C12 acetate ester and/or a C14 acetate ester and/or a C16 acetate ester. In some embodiments, the C12 acetate ester and/or C14 acetate ester and/or C16 acetate ester is monounsaturated (i.e., comprises one alkene group). In some embodiments, the second active component comprises Z9-14:OAc, Z7-12:OAc, Z9-12:OAc, Z11-16:OAc or any combination thereof. In some embodiments, the second active component comprises at least two of Z9-14:OAc, Z7-12:OAc, Z9-12:OAc, and Z11-16:OAc. In some embodiments, the second active component comprises at least one or at least two of Z9-14:OAc, Z7-12:OAc, Z9-12:OAc, and Z11-16:OAc and one or more additional component selected from the group comprising Z10-14:OAc, 14:OAc, Z11-14:OAc, E7-12:OAc, 12:OAc, Z11-12:OAc, Z9-14:Ald, and Z11-16:Ald.

In some embodiments, one or both of the first and second active components further includes, in addition to one or more pheromone, a carrier. The carrier can be, but is not limited to, an inert liquid or solid. Exemplary solid carriers include, but are not limited to, fillers such as kaolin, bentonite, dolomite, calcium carbonate, talc, powdered magnesia, Fuller's earth, wax, gypsum, diatomaceous earth, rubber, plastic, China clay, mineral earths such as silicas, silica gels, silicates, attaclay, limestone, chalk, loess, clay, dolomite, calcium sulfate, magnesium sulfate, magnesium oxide, ground synthetic materials, fertilizers such as ammonium sulfate, ammonium phosphate, ammonium nitrate, thiourea and urea, products of vegetable origin such as cereal meals, tree bark meal, wood meal and nutshell meal, cellulose powders, attapulgites, montmorillonites, mica, vermiculites, synthetic silicas and synthetic calcium silicates, or compositions of these. Exemplary liquid carriers include, but are not limited to, water; alcohols, such as ethanol, butanol or glycol, as well as their ethers or esters, such as methylglycol acetate; ketones, such as acetone, cyclohexanone, methylethyl ketone, methylisobutylketone, or isophorone; alkanes such as hexane, pentane, or heptanes; aromatic hydrocarbons, such as xylenes or alkyl naphthalenes; mineral or vegetable oils; aliphatic chlorinated hydrocarbons, such as trichloroethane or methylene chloride; aromatic chlorinated hydrocarbons, such as chlorobenzenes; water-soluble or strongly polar solvents such as dimethylformamide, dimethyl sulfoxide, or N-methylpyrrolidone; liquefied gases; waxes, such as beeswax, lanolin, shellac wax, carnauba wax, fruit wax (such as bayberry or sugar cane wax) candelilla wax, other waxes such as microcrystalline, ozocerite, ceresin, or montan; salts such as monoethanolamine salt, sodium sulfate, potassium sulfate, sodium chloride, potassium chloride, sodium acetate, ammonium hydrogen sulfate, ammonium chloride, ammonium acetate, ammonium formate, ammonium oxalate, ammonium carbonate, ammonium hydrogen carbonate, ammonium thiosulfate, ammonium hydrogen diphosphate, ammonium dihydrogen monophosphate, ammonium sodium hydrogen phosphate, ammonium thiocyanate, ammonium sulfamate or ammonium carbamate and mixtures thereof. Baits or feeding stimulants can also be added to the carrier.

In some embodiments, the first active component and the second active component are separately formulated. In some embodiments, the first active component and the second active component are provided in the same formulation. In some embodiments, one or both of the first and the second active component is formulated in a slow release formulation, so as to provide slow release of the component into the atmosphere and/or so as to be protected from degradation following release. For example, the pheromone composition or individual components thereof can be formulated in carriers such as microcapsules, biodegradable flakes and paraffin wax-based matrices. Alternatively, the pheromone composition or individual components thereof can be formulated as a slow release sprayable.

In some embodiments, the presently disclosed composition or individual components thereof can include one or more polymeric agents known to one skilled in the art. The polymeric agents can control the rate of release of the composition to the environment. In some embodiments, the polymeric agent-containing composition is impervious to environmental conditions. The polymeric agent can also be a sustained-release agent that enables the composition to be released to the environment in a sustained manner. Examples of polymeric agents include, but are not limited to, celluloses, proteins such as casein, fluorocarbon-based polymers, hydrogenated rosins, lignins, melamine, polyurethanes, vinyl polymers such as polyvinyl acetate (PVAC), polycarbonates, polyvinylidene dinitrile, polyamides, polyvinyl alcohol (PVA), polyamide-aldehyde, polyvinyl aldehyde, polyesters, polyvinyl chloride (PVC), polyethylenes, polystyrenes, polyvinylidene, silicones, and combinations thereof. Examples of celluloses include, but are not limited to, methylcellulose, ethyl cellulose, cellulose acetate, cellulose acetate-butyrate, cellulose acetate-propionate, cellulose propionate, and combinations thereof. In some embodiments, the presently disclosed composition or individual components thereof can be formulated as a microencapsulated pheromone, in which small droplets of one or more active component are enclosed within polymer capsules. The capsules can control the release rate of the pheromone active component into the surrounding environment and can be small enough to be applied in the same method as used to spray insecticides. The effective field longevity of the microencapsulated pheromone formulations can range from a few days to slightly more than a week, depending on climatic conditions, capsule size and chemical properties. Other agents which can be used in slow-release or sustained-release formulations include fatty acid esters (such as a sebacate, laurate, palmitate, stearate or arachidate ester) and fatty alcohols (such as undecanol, dodecanol, tridecanol, tridecenol, tetradecanol, tetradecenol, tetradecadienol, pentadecanol, pentadecenol, hexadecanol, hexadecenol, hexadecadienol, octadecenol and octadecadienol).

The aldehyde group in compounds such as nonanal can react with abiotic environmental factors (e.g., oxygen and water) to form other chemical groups (e.g., acids and alcohols). Thus, nonanal can be sensitive to chemical degradation that can reduce its activity as a FAW attractant. In addition, nonanal and other C7-C11 aldehydes are more volatile than the previously identified FAW pheromone components. Therefore, in some embodiments, e.g., to compensate for higher volatility and to protect the C7-C11 aldehyde from degradation, it can be treated differently (e.g., formulated differently) from other pheromone components in a lure or trap. For example, in some embodiments, the C7-C11 aldehyde (i.e., the first active component) is placed in a separate dispenser next another pheromone dispenser comprising a C12-C16 acetate ester, aldehyde, or primary alcohol (such as a commercial pheromone dispenser for FAW currently on the market).

In some embodiments, the first active component (i.e., the C7-C11 aldehyde) is formulated for slow release. In some embodiments, the first active component is formulated in an oil. In some embodiments, the oil is paraffin oil or another non-volatile and odorless oil. In some embodiments, the oil formulation of the first active component is placed in a dispenser or container protected from light. For instance, because of the high affinity of nonanal to paraffin oil, which is a mixture of alkanes, the emission of nonanal is reduced and prolonged.

In some embodiments, the first active component comprises, consists essentially of, or consists of nonanal and the second active component comprises, consists essentially of, or consists of Z9-14:OAc. In some embodiments, the nonanal is present at an amount ranging from about 0.10 weight (wt) % to about 50 wt % (e.g., about 0.10, 0.20, 0.30, 0.40, 0.50. 0.60, 0.70, 0.80, 0.90, 1.0, 2.0, 3.0, 4.0, 5.0, 6.0, 7.0, 8.0, 9.0, 10, 15, 20, 25, 30, 35, 40, 45, or about 50 wt %) compared to the weight of the Z9-14:OAc. In some embodiments, the nonanal is present at about 0.10 wt % to about 10 wt % (e.g., about 0.10, 0.50, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or about 10 wt %) compared to the weight of the Z9-14:OAc. In some embodiments, the nonanal is present at about 0.50 wt % to about 5 wt % compared to the weight of the Z9-14:OAc. In some embodiments, the nonanal is present at about 1 wt % compared to the weight of the Z9-14:OAc.

Thus, in some embodiments, at least Z7-12:OAc is provided as the second active component in combination with a first component comprising nonanal and/or another C7-C11 aldehyde. In some embodiments, the presently disclosed attractant compositions can comprise a combination that comprises at least nonanal (and/or another C7-C11 aldehyde), Z9-14:OAc, and Z7-12:OAc. In some embodiments, the compositions further comprise additional known FAW pheromone components (e.g., at least one or more additional or at least two or more additional known FAW pheromone components), such as those described hereinabove. In some embodiments, the compositions further comprise additional biosynthetic precursors of known FAW pheromone components and/or additional known Spodoptera pheromone components (e.g., C12-C16 primary alcohols, such as C12-C16 monounsaturated primary alcohols). In some embodiments, the presently disclosed subject matter provides a combination of nonanal (and/or another C1-C11, aldehyde) and three or four (or more) previously known FAW/Spodoptera pheromone components (or biosynthetic precursors thereof).

In some embodiments, the composition further comprises one or more non-pheromone insect control agent, such as a pest killing agent, a slow-acting insecticide, or a biological agent. For example, the biological agent can be selected from a bacteria, a fungi, a virus, and a nematode. In some embodiments, the pest killing agent is a fast-acting insecticide. Examples of the chemical insecticides include, but are not limited to, Chemical insecticides include, but are not limited to, abamectin, AC 303 630, acephate, acrinathrin, alanycarb, aldicarb, alphamethrin, amitraz, avermectin, AZ 60541, azadirachtin, azinphos A, azinphos M, azocyclotin, bendiocarb, benfuracarb, bensultap, betacyfluthrin, bifenthrin, bioresmethrin, BPMC, brofenprox, bromophos A, bufencarb, buprofezin, butocarboxin, butylpyridaben, cadusafos, carbaryl, carbofuran, carbophenothion, carbosulfan, cartap, CGA 157 419, CGA 184699, chloethocarb, chlorethoxyfos, chlorfenvinphos, chlorfluazuron, chlormephos, chlorpyrifos, chlorpyrifos M, cis-Resmethrin, clocythrin, clofentezine, cyanophos, cycloprothrin, cyfluthrin, cyhalothrin, cyhexatin, cypermethrin, cyromazine, deltamethrin, demeton M, demeton S, demeton-S-methyl, diafenthiuron, diazinon, dichlofenthion, dichlorvos, dicliphos, dicrotophos, diethion, diflubenzuron, dimethoate, dimethylvinphos, dioxathion, disulfoton, edifenphos, emamectin, esfenvalerate, ethiofencarb, ethion, ethofenprox, ethoprophos, etrimphos, fenamiphos, fenazaquin, fenbutatin oxide, fenitrothion, fenobucarb, fenothiocarb, fenoxycarb, fenpropathrin, fenpyrad, fenpyroximate, fenthion, fenvalerate, fipronil, fluazinam, flucycloxuron, flucythrinate, flufenoxuron, flufenprox, fluvalinate, fonophos, formothion, fosthiazate, fubfenprox, furathiocarb, HCH, heptenophos, hexaflumuron, hexythiazox, imidacloprid, iprobenfos, isazophos, isofenphos, isoprocarb, isoxathion, ivermectin, lambda-cyhalothrin, lufenuron, malathion, mecarbam, mervinphos, mesulfenphos, metaldehyde, methacrifos, methamidophos, methidathion, methiocarb, methomyl, metolcarb, milbemectin, monocrotophos, moxidectin, naled, NC 184, NI 25, nitenpyram omethoat, oxamyl, oxydemethon M, oxydeprofos, parathion A, parathion M, permethrin, phenothrin, phenthoate, phorate, phosalone, phosmet, phosphamidon, phoxim, pirimicarb, pirimiphos M, pirimiphos A, profenofos, promecarb, propaphos, propoxur, prothiofos, prothoate, pymetrozin, pyrachlophos, pyridaphenthion, pyresmethrin, pyrethrum, pyridaben, pyrimidifen, pyriproxifen, quinalphos, resmethrin, RH 5992, salithion, sebufos, silafluofen, sulfotep, sulprofos, tebufenozid, tebufenpyrad, tebupirimiphos, teflubenzuron, tefluthrin, temephos, terbam, terbufos, tetrachlorvinphos, thiafenox, thiodicarb, thiofanox, thiomethon, thionazin, thuringiensin, tralocytrin, tralomethrin, triarathen, triazophos, triazuron, trichlorfon, triflumuron, trimethacarb, transfluthrin vamidothion, XMC, xylylcarb, and zetamethrin. Examples of the biological insecticides include, but are not limited to, azadirachtin (neem oil), toxins from natural pyrethrins, Bacillus thuringiensis and Beauveria bassiana, viruses, and peptides.

In some embodiments, one or both component of the presently disclosed composition (e.g., the first and/or second active component(s)) can be formulated with a synergist. The term, “synergist,” as used herein, refers to a substance that can be used for reducing the dose or enhancing the effectiveness of the first and/or second active component for attracting at least one Spodoptera species of agricultural pest. In some embodiments, the synergist can be an independent attractant of an insect in the absence of a pheromone. In some embodiments, the synergist is not an independent attractant of an insect in the absence of a pheromone. In some embodiments, the synergist is a volatile phytochemical that attracts at least one species of Spodoptera (e.g., FAW). The term, “phytochemical,” as used herein, refers to a compound occurring naturally in a plant species. In some embodiments, the synergist is selected from the group comprising β-caryophyllene, iso-caryophyllene, α-humulene, inalool, Z3-hexenol/yl acetate, β-farnesene, benzaldehyde, phenylacetaldehyde, and combinations thereof.

In some embodiments, the composition can further include one or more optional adjuvants and/or other compounds, provided that such optional adjuvants or other compounds do not substantially interfere with the activity of active components. In some embodiments, the optional adjuvants and/or other compounds can be selected from the group including, but not limited to: wetters, compatibilizing agents (also referred to as “compatibility agents”), antifoam agents, cleaning agents, sequestering agents, drift reduction agents, neutralizing agents and buffers, corrosion inhibitors, dyes, odorants, spreading agents (also referred to as “spreaders”), penetration aids (also referred to as “penetrants”), sticking agents (also referred to as “stickers” or “binders”), dispersing agents, fillers, thickening agents (also referred to as “thickeners”), stabilizers, emulsifiers, freezing point depressants, antimicrobial agents, and the like. Examples of stabilizers include, but are not limited to, fatty acids and vegetable oils, such as for example olive oil, soybean oil, corn oil, safflower oil, canola oil, and combinations thereof. Examples of fillers include, but are not limited to, one or more mineral clays (e.g., attapulgite). In some embodiments, the filler is an organic thickener. Examples of such thickeners include, but are not limited to, methyl cellulose, ethyl cellulose, and any combinations thereof.

In some embodiments, the composition can include one or more solvents. Compositions containing solvents are desirable when a user is to employ liquid compositions which can be applied by brushing, dipping, rolling, spraying, or otherwise applying the liquid compositions to substrates on which the user wishes to provide a pheromone coating (e.g., a lure). In some embodiments, the solvent(s) to be used is/are selected so as to solubilize, or substantially solubilize, the one or more ingredients (e.g., one or more active components, or individual compounds thereof) of the composition. Examples of solvents include, but are not limited to, water, aqueous solvent (e.g., mixture of water and ethanol), ethanol, methanol, chlorinated hydrocarbons, petroleum solvents, turpentine, xylene, and any combinations thereof.

In some embodiments, the presently disclosed composition can comprise one or more organic solvents. Organic solvents can be used, for example, in the formulation of emulsifiable concentrates, ULV formulations, and to a lesser extent granular formulations. Sometimes mixtures of solvents are used. In some embodiments, the organic solvents can include aliphatic paraffinic oils such as kerosene or refined paraffins. In come embodiments, the organic solvents can comprise an aromatic solvent such as xylene and higher molecular weight fractions of C9 and C10 aromatic solvents. In some embodiments, chlorinated hydrocarbons are useful as co-solvents to prevent crystallization when the formulation is emulsified into water. In some embodiments, alcohols can be used as co-solvents to increase solvent power.

In some embodiments, the presently disclosed composition can comprise one or more solubilizing agents. Solubilizing agents include surfactants, which can form micelles in water at concentrations above the critical micelle concentration. The micelles are then able to dissolve or solubilize water-insoluble materials inside the hydrophobic part of the micelles. In some embodiments, the surfactants are non-ionics, e.g., sorbitan monooleates; sorbitan monooleate ethoxylates; and methyl oleate esters.

In some embodiments, the presently disclosed composition can comprise one or more binders. Binders can be used to promote association of the composition with the surface of the material on which said composition is coated. In some embodiments, the binder can be used to promote association of another additive (e.g., insecticide, insect growth regulators, and the like) to the first and/or second active component of the composition and/or the surface of a material. For example, a binder can include a synthetic or natural resin typically used in paints and coatings. These can be modified to cause the coated surface to be friable enough to allow insects to bite off and ingest the components of the composition (e.g., insecticide, insect growth regulators, and the like), while still maintaining the structural integrity of the coating.

Non-limiting examples of binders include polyvinylpyrrolidone, polyvinyl alcohol, partially hydrolyzed polyvinyl acetate, carboxymethylcellulose, starch, vinylpyrrolidone/vinyl acetate copolymers and polyvinyl acetate, or compositions of these; lubricants such as magnesium stearate, sodium stearate, talc or polyethylene glycol, or compositions of these; antifoams such as silicone emulsions, long-chain alcohols, phosphoric esters, acetylene diols, fatty acids or organofluorine compounds, and complexing agents such as: salts of ethylenediaminetetraacetic acid (EDTA), salts of trinitrilotriacetic acid or salts of polyphosphoric acids, or compositions of these. In some embodiments, the binder also acts a filler and/or a thickener. Examples of such binders include, but are not limited to, one or more of shellac, acrylics, epoxies, alkyds, polyurethanes, linseed oil, tung oil, and any combinations thereof.

In some embodiments, the presently disclosed composition can comprise one or more surface-active agents. In some embodiments, the surface-active agents are added to a liquid composition of the presently disclosed subject matter. In some embodiments, the surface-active agents are added to solid formulations, e.g., those designed to be diluted with a carrier before application. Thus, in some embodiments, the presently disclosed composition comprises one or more surfactants. Surfactants are sometimes used, either alone or with other additives, such as mineral or vegetable oils as adjuvants to spray-tank mixes to improve the biological performance of the composition on the target. The surface-active agents can be anionic, cationic, or nonionic in character, and can be employed as emulsifying agents, wetting agents, suspending agents, or for other purposes. In some embodiments, the surfactants are non-ionics such as: alky ethoxylates, linear aliphatic alcohol ethoxylates, and aliphatic amine ethoxylates. In some embodiments, the surfactants can include alkali metal, alkaline earth metal or ammonium salts of aromatic sulfonic acids, for example, ligno-, phenol-, naphthalene- and dibutylnaphthalenesulfonic acid, and of fatty acids of arylsulfonates, of alkyl ethers, of lauryl ethers, of fatty alcohol sulfates and of fatty alcohol glycol ether sulfates, condensates of sulfonated naphthalene and its derivatives with formaldehyde, condensates of naphthalene or of the naphthalenesulfonic acids with phenol and formaldehyde, condensates of phenol or phenolsulfonic acid with formaldehyde, condensates of phenol with formaldehyde and sodium sulfite, polyoxyethylene octylphenyl ether, ethoxylated isooctyl-, octyl- or nonylphenol, tributylphenyl polyglycol ether, alkylaryl polyether alcohols, isotridecyl alcohol, ethoxylated castor oil, ethoxylated triarylphenols, salts of phosphated triarylphenolethoxylates, lauryl alcohol polyglycol ether acetate, sorbitol esters, lignin-sulfite waste liquors or methylcellulose, or compositions of these.

In some embodiments, the surface-active agent(s) can include salts of alkyl sulfates, such as diethanolammonium lauryl sulfate; alkylarylsulfonate salts, such as calcium dodecylbenzenesulfonate; alkylphenol-alkylene oxide addition products, such as nonylphenol-C18 ethoxylate; alcohol-alkylene oxide addition products, such as tridecyl alcohol-C16 ethoxylate; soaps, such as sodium stearate; alkylnaphthalene-sulfonate salts, such as sodium dibutyl-naphthalenesulfonate; dialkyl esters of sulfosuccinate salts, such as sodium di(2-ethylhexyl)sulfosuccinate; sorbitol esters, such as sorbitol oleate; quaternary amines, such as lauryl trimethylammonium chloride; polyethylene glycol esters of fatty acids, such as polyethylene glycol stearate; block copolymers of ethylene oxide and propylene oxide; salts of mono and dialkyl phosphate esters; vegetable oils such as soybean oil, rapeseed/canola oil, olive oil, castor oil, sunflower seed oil, coconut oil, corn oil, cottonseed oil, linseed oil, palm oil, peanut oil, safflower oil, sesame oil, tung oil and the like; and esters of the above vegetable oils, particularly methyl esters.

In some embodiments, the presently disclosed composition can comprise one or more wetting agents. A wetting agent is a substance that when added to a liquid increases the spreading or penetration power of the liquid by reducing the interfacial tension between the liquid and the surface on which it is spreading. Wetting agents can be used in agrochemical formulations during processing and manufacture to increase the rate of wetting of powders in water to make concentrates for soluble liquids or suspension concentrates; and/or during mixing of a product with water in a spray tank or other vessel to reduce the wetting time of wettable powders and to improve the penetration of water into water-dispersible granules. In some embodiments, examples of wetting agents used in the presently disclosed composition (or a component thereof), include wettable powders, suspension concentrates, and water-dispersible granule formulations are: sodium lauryl sulphate; sodium dioctyl sulphosuccinate; alkyl phenol ethoxylates; and aliphatic alcohol ethoxylates.

In some embodiments, the presently disclosed composition comprises one or more dispersing agents. A dispersing agent is a substance which adsorbs onto the surface of particles and helps to preserve the state of dispersion of the particles and prevents them from reaggregating. In some embodiments, dispersing agents are added to a composition of the presently disclosed subject matter to facilitate dispersion and suspension during manufacture, and to ensure the particles redisperse into water in a spray tank. In some embodiments, dispersing agents are used in wettable powders, suspension concentrates, and water-dispersible granules. Surfactants that are used as dispersing agents have the ability to adsorb strongly onto a particle surface and provide a charged or steric barrier to re-aggregation of particles. In some embodiments, the surfactants are anionic, non-ionic, or mixtures of the two types.

In some embodiments, for wettable powder formulations, the dispersing agents comprise one or more sodium lignosulphonates. In some embodiments, suspension concentrates provide good adsorption and stabilization using polyelectrolytes, such as sodium naphthalene sulphonate formaldehyde condensates. In some embodiments, tristyrylphenol ethoxylated phosphate esters are used. In some embodiments, alkylarylethylene oxide condensates and EO-PO block copolymers are combined with anionics as dispersing agents for suspension concentrates.

In some embodiments, the presently disclosed composition can comprise one or more polymeric surfactants. In some embodiments, the polymeric surfactants have very long hydrophobic ‘backbones’ and a large number of ethylene oxide chains forming the ‘teeth’ of a ‘comb’ surfactant. In some embodiments, these high molecular weight polymers can give good long-term stability to suspension concentrates, because the hydrophobic backbones have many anchoring points onto the particle surfaces. In some embodiments, the dispersing agents are selected from: sodium lignosulphonates; sodium naphthalene sulphonate formaldehyde condensates; tristyrylphenol ethoxylate phosphate esters; aliphatic alcohol ethoxylates; alky ethoxylates; EO-PO block copolymers; and graft copolymers.

In some embodiments, the presently disclosed compositions can comprise one or more emulsifying agents. An emulsifying agent is a substance, which stabilizes a suspension of droplets of one liquid phase in another liquid phase. Without the emulsifying agent the two liquids would separate into two immiscible liquid phases. In some embodiments, the emulsifier comprises an alkylphenol or aliphatic alcohol with about 12 or more ethylene oxide units and the oil-soluble calcium salt of dodecylbenzene sulphonic acid. A range of hydrophile-lipophile balance (“HLB”) values from 8 to 18 can normally provide good stable emulsions. In some embodiments, emulsion stability can sometimes be improved by the addition of a small amount of an EO-PO block copolymer surfactant.

In some embodiments, the presently disclosed compositions (or a formulation comprising the first or second active component thereof) can comprise one or more gelling agents. Thickeners or gelling agents can be used in the formulation of suspension concentrates, emulsions, and suspoemulsions to modify the rheology or flow properties of the liquid and to prevent separation and settling of the dispersed particles or droplets. Thickening, gelling, and anti-settling agents generally can fall into two categories: water-insoluble particulates and water-soluble polymers. It is possible to produce suspension concentrate formulations using clays and silicas. In some embodiments, the presently disclosed compositions comprise one or more thickeners including, but not limited to: montmorillonite, e.g. bentonite; magnesium aluminum silicate; and attapulgite. In some embodiments, a polysaccharide can be used as a thickening agent. The types of polysaccharides typically used as thickening agents are natural extracts of seeds and seaweeds or synthetic derivatives of cellulose. In some embodiments the thickening agent comprises xanthan and/or cellulose. In some embodiments, the thickening agents can be selected from the group including, but not limited to, guar gum; locust bean gum; carrageenam; alginates; methyl cellulose; sodium carboxymethyl cellulose (SCMC); hydroxyethyl cellulose (HEC). In some embodiments, the compositions of the presently disclosed subject matter can include one or more other types of anti-settling agents, such as modified starches, polyacrylates, polyvinyl alcohol, xanthan gum, and polyethylene oxide.

In some embodiments, the presence of surfactants, which lower interfacial tension, can cause water-based formulations to foam during mixing operations in production and in application through a spray tank. Thus, in some embodiments, in order to reduce the tendency to foam, anti-foam agents are often added either during the production stage or before filling into bottles/spray tanks. Generally, there are two types of anti-foam agents, silicones and nonsilicones. Silicones are usually aqueous emulsions of dimethyl polysiloxane, while the nonsilicone anti-foam agents are water-insoluble oils, such as octanol and nonanol, or silica. In both cases, the function of the anti-foam agent is to displace the surfactant from the air-water interface.

In some embodiments, the presently disclosed composition can comprise a preservative.

In some embodiments, the composition of the presently disclosed subject matter can include one or more insect feeding stimulants. Examples of insect feeding stimulants include, but are not limited to, crude cottonseed oil, fatty acid esters of phytol, fatty acid esters of geranyl geraniol, fatty acid esters of other plant alcohols, plant extracts, and combinations thereof.

In some embodiments, the composition can include one or more insect growth regulators (“IGRs”). IGRs can be used to alter the growth of the insect and produce deformed insects. Examples of insect growth regulators include, for example, dimilin. In some embodiments, the composition can include one or more insect sterilants that sterilize trapped insects or otherwise block their reproductive capacity, thereby reducing the population in the following generation. In some embodiments, allowing the sterilized insects to survive and compete with non-trapped insects for mates is more effective than killing them outright.

In some embodiments, the compositions disclosed herein (or one of the active components thereof) can be formulated as a sprayable composition (i.e., a sprayable pheromone composition). An aqueous solvent can be used in the sprayable composition, e.g., water or a mixture of water and an alcohol, glycol, ketone, or other water-miscible solvent. In some embodiments, the water content of such mixture is at least about 10%, at least about 20%, at least about 30%, at least about 40%, 50%, at least about 60%, at least about 70%, at least about 80%, or at least about 90%. In some embodiments, the sprayable composition is a concentrate, i.e. a concentrated suspension of the first and/or second active component(s), and other additives (e.g., a waxy substance, a stabilizer, and the like) in the aqueous solvent, and can be diluted to the final use concentration by addition of solvent (e.g., water).

In some embodiments, a waxy substance can be used as a carrier for the first and/or second active component in the sprayable composition. The waxy substance can be, e.g., a biodegradable wax, such as bees wax, carnauba wax and the like, candelilla wax (hydrocarbon wax), montan wax, shellac and similar waxes, saturated or unsaturated fatty acids, such as lauric, palmitic, oleic or stearic acid, fatty acid amides and esters, hydroxylic fatty acid esters, such as hydroxyethyl or hydroxypropyl fatty acid esters, fatty alcohols, and low molecular weight polyesters such as polyalkylene succinates.

In some embodiments, a stabilizer can be used with the sprayable compositions. The stabilizer can be used to regulate the particle size of concentrate and/or to allow the preparation of a stable suspension of the composition. In some embodiments, the stabilizer is selected from hydroxylic and/or ethoxylated polymers. Examples include ethylene oxide and propylene oxide copolymer, polyalcohols, including starch, maltodextrin and other soluble carbohydrates or their ethers or esters, cellulose ethers, gelatin, polyacrylic acid and salts and partial esters thereof and the like. In other embodiments, the stabilizer can include polyvinyl alcohols and copolymers thereof, such as partly hydrolyzed polyvinyl acetate. The stabilizer may be used at a level sufficient to regulate particle size and/or to prepare a stable suspension, e.g., between 0.1% and 15% of the aqueous solution.

In some embodiments, a binder can be used with the sprayable compositions. In some embodiments, the binder can act to further stabilize the dispersion and/or improve the adhesion of the sprayed dispersion to the target locus (e.g., a trap, lure, plant, etc.). The binder can be a polysaccharide, such as an alginate, cellulose derivative (acetate, alkyl, carboxymethyl, hydroxyalkyl), starch or starch derivative, dextrin, gum (arabic, guar, locust bean, tragacanth, carrageenan, and the like), sucrose, and the like. The binder can also be a non-carbohydrate, water-soluble polymer such as polyvinyl pyrrolidone, or an acidic polymer such as polyacrylic acid or polymethacrylic acid, in acid and/or salt form, or mixtures of such polymers.

In some embodiments, the presently disclosed composition can be used in conjunction with a dispenser for release of the composition or the individual active components in a particular environment. Any suitable dispenser known in the art can be used. Examples of such dispensers include but are not limited to, aerosol emitters, hand-applied dispensers, bubble caps comprising a reservoir with a permeable barrier through which pheromones are slowly released, pads, beads, tubes rods, spirals or balls composed of rubber, plastic, leather, cotton, cotton wool, wood or wood products that are impregnated with the composition. For example, polyvinyl chloride laminates, pellets, granules, ropes or spirals from which the composition evaporates, or rubber septa. One of skill in the art will be able to select suitable carriers and/or dispensers for the desired mode of application, storage, transport or handling.

Accordingly, in some embodiments, the composition of the presently disclosed subject matter can be coated on or sprayed on a solid substrate that can be used as a dispenser comprising, for example, a polymer, a glass, a rubber, an elastomer, cellulose, wood, and felt, or the substrate can be otherwise impregnated with a composition of the presently disclosed subject matter or a first or second active component thereof.

In another embodiment, a dispenser can be used that contaminates the male insects with a powder containing the active components. The contaminated males then fly off and provide a source of mating disruption by permeating the atmosphere with the active pheromone components, or by attracting other males to the contaminated males, rather than to real females.

The first and second active components can be provided in the same or separate dispensers. In some embodiments, the first active component is provided in a separate dispenser from the second active component. In some embodiments, the first active component is formulated in an oil and provided in a first dispenser and the second active component is provided in a second dispenser. In some embodiments, the second dispenser comprises rubber (e.g., a rubber septa).

In some embodiments, the C7-C11 aldehyde is formulated in a slow release formulation. In some embodiments, nonanal can be diluted in an oil carrier, like paraffin oil, or another non-volatile and odorless oil, and placed in a dispenser or container protected from light. For instance, because of the high affinity of nonanal to paraffin oil, which is a mixture of alkanes, the emission of nonanal is reduced and prolonged.

In some embodiments, the presently disclosed subject matter provides a nonanal dispenser (or other C7-C11 aldehyde dispenser) comprising a glass (e.g., borosilicate) vial (e.g., a 2 mL vial) covered with aluminum foil or another material that can block sunlight. The vial can further contain deactivated glass wool (e.g., about 50 mg of deactivated glass wool) or another fiberous substrate. The aluminum foil or other covering material can prevent the exposure of nonanal to sunlight, which can facilitate chemical reactions with environmental factors. The glass wool or other fiberous substrate can increase the surface of the nonanal solution and hold the solution in place inside the vial. Once the nonanal solution is loaded in the vial, the vial can be capped. For example, in some embodiments, the cap has a silicone septum with a PTFE liner through which a microcapillary glass is inserted (e.g., having a 1.25 inch; O.D.: 0.034 inch; I.D.: 0.0157 inch; 4 μL internal volume) to allow the volatile nonanal to evaporate from the vial.

As described in the Examples below, the nonanal percentage (compared to the main pheromone component, e.g., Z9-14:OAc, which is typically present at about 1 to 10 mg) that rendered the highest number of trap catches in the field is 1% (weight/weight). However, studies showed that the optimal percentage can cover a broader range. Furthermore, as would be understood by one of ordinary skill in the art, the optimal percentage can vary depending on how the nonanal is formulated for its slow release. Therefore, in some embodiments, the presently disclosed subject matter provides compositions (e.g., FAW lures) that comprise between about 0.10 wt % to about 50 wt. % nonanal compared to the amount of Z9-14:OAc. In some embodiments, the composition comprises, in addition to nonanal (and/or another C7-C10 aldehyde), Z9-14:OAc (e.g., 98.44 wt %) and Z7-12:OAc (0.57 wt %). In some embodiments, the composition further comprises one or more or two or more additional known FAW/Spodoptera pheromone components, such as one of the other known FAW/Spodoptera pheromone components described hereinabove. For instance, Z9-12:OAc and Z11-16:OAc are typically formulated into four component commercial lures and can also be included in the instant compositions (e.g., as part of a composition also comprising nonanal (and/or another C7-C11 aldehyde), Z9-14:OAc, and Z7-12:OAc).

The amount of nonanal can be modified depending upon the type of material used to dispense the pheromones. For instance, many pheromone dispensors are made of rubber septa and nonanal is released relatively rapidly from this material, unlike the other pheromone compounds. In some embodiments, the nonanal and/or other C7-C11 aldehyde can be provided in a dispenser that slowly and constantly releases the aldehyde. In such embodiments, the amount of nonanal (and/or other C7-C11 aldehyde) can be increased to last longer. For example, in some embodiments, the nonanal (and/or other C7-C11 aldehyde) can be formulated to continuously dispense the aldehyde for between about 2 weeks and about 2 months. In some embodiments, aldehyde can be formulated to continuously dispense for over two months.

In some embodiments, the lures or dispensors comprising nonanal can be used for surveillance and/or monitoring of FAW or another Spodoptera species. In some embodiments, the lures or dispensors can be used to control FAW or other Spodoptera species, e.g., via an “attract-and-kill” approach, via mass trapping, via mating disruption or via an autoinoculation approach.

To elaborate, the deployment of traps with synthetic lures that mimic the pest's sex pheromone provides an approach to detect new agricultural insect pest infestations at early stages, monitor established infestations, and control resurgent pest populations. Sex pheromones are good attractants for pest management because males are highly mobile and respond to extremely low amounts of highly species-specific sex pheromone. In addition, they reliably predict when adult insect pests fly and adult insects are much more accessible and susceptible to insecticide and biocide treatments than are larval stages. Further, at high doses, sex pheromones are effective at suppressing mating, a process called ‘mating disruption’, that effectively controls pest populations with minimal inputs.

Accordingly, in some embodiments, the composition (or lures or dispensers comprising one or more components of the presently disclosed composition) can be incorporated into a trap or multi-component device, comprising a housing for trapping insects or insect pests. In some embodiments, the presently disclosed composition can be used in a trap commonly used to attract any insect species, e.g., Spodoptera species insects. Such traps are well known to one skilled in the art and are commonly used in many states and countries in insect eradication programs. Such traps can have any design, such as, but not limited to bucket-style traps (e.g., a Unitrap, available from Great Lakes IPM, Vestabury, Mich., United States of America), sleeve-style traps, Hartstack traps, and sticky traps (e.g., plastic delta-shaped houslings with sticky liners inserted therein). Additional types of traps are described, for example, in Cork (“A Pheromone Manual”, Natural Resources Institute, Chatham Maritime ME4 4TB, UK (2004)).

In some embodiments, the trap includes one or more septa, containers, or storage receptacles for holding the composition. Thus, in some embodiments, the presently disclosed subject matter provides a trap loaded with the presently disclosed two component composition. The traps can be used, for example, to attract insects as part of a strategy for insect monitoring, mass trapping, mating disruption, or lure/attract and kill for example by incorporating a toxic substance into the trap to kill insects caught.

Mass trapping can involve placing a high density of traps in a crop to be protected so that a high proportion of the insects are removed before the crop is damaged. Lure/attract-and-kill techniques are similar except once the insect is attracted to a lure, it is subjected to a killing agent. Where the killing agent is an insecticide, a dispenser can also contain a bait or feeding stimulant that can entice the insects to ingest an effective amount of an insecticide. The insecticide can be an insecticide known to one skilled in the art. The insecticide can be mixed with the composition of the presently disclosed subject matter (or a single component thereof) or be separately present in a trap. Such traps can take any suitable form, and killing traps need not necessarily incorporate toxic substances, the insects being optionally killed by other means, such as drowning or electrocution. Alternatively, the traps can contaminate the insect with a fungus or virus that kills the insect later. Even where the insects are not killed, the trap can serve to remove the male insects from the locale of the female insects, to prevent breeding.

In some embodiments, the trap is selected from the group including, but not limited to, water traps, sticky traps, and one-way traps. Sticky traps come in many varieties. One example of a sticky trap is of cardboard construction, triangular or wedge-shaped in cross-section, where the interior surfaces are coated with a non-drying sticky substance. The insects contact the sticky surface and are caught. Water traps include pans of water and detergent that are used to trap insects. The detergent destroys the surface tension of the water, causing insects that are attracted to the pan, to drown in the water. One-way traps allow an insect to enter the trap but prevent it from exiting. In some embodiments, the traps can be colored brightly, to provide additional attraction for the insects.

In some embodiments, the traps containing the presently disclosed composition can be combined with other kinds of trapping mechanisms. For example, in addition to the presently disclosed composition, the trap can include one or more florescent lights, one or more sticky substrates and/or one or more colored surfaces for attracting insects. In other embodiments, the trap containing the presently disclosed composition does not have other kinds of trapping mechanisms.

The trap can be set at any time of the year in a field. Those of skill in the art can readily determine an appropriate amount of the compositions to use in a particular trap and can also determine an appropriate density of traps/acre of crop field to be protected. The trap can be positioned in an area infested (or potentially infested) with insects. In some embodiments, the trap is placed on or close to a tree or plant. The aroma of the pheromones in the presently disclosed composition (i.e., the first and/or second active components) can attract the insects to the trap. The insects can then be caught, immobilized and/or killed within the trap, for example, by a killing agent present in the trap.

In some embodiments, one or more traps can be placed within a field or orchard or other local to overwhelm the pheromones emitted by the females, so that the males simply cannot locate the females. In this respect, a trap can be a simple apparatus, for example, a protected wickable to dispense first and second active components.

The traps can be provided in made-up form, where the presently disclosed composition has already been applied. In such an instance, depending on the half-life of the active components in the composition, the active components can be exposed or can be sealed in a conventional manner, such as is standard with other aromatic dispensers, the seal only being removed once the trap is in place. Alternatively, the trap can be provided separately from the composition and the composition can be provided in a dispensable format so that an amount can be applied to trap, once the trap is in place. In some embodiments, the presently disclosed composition can be provided in in a sachet or other dispenser or a kit comprising a sachet or other dispenser for each of the two active components.

Accordingly, in some embodiments, the presently disclosed subject matter provides a multi-component device (i.e., a “trap”) for attracting and capturing male Spodoptera frugiperda or males of another Spodoptera species, wherein the trap comprises a first active component comprising at least one C7-C11 aldehyde, a second active component comprising at least one saturated or unsaturated C12-C16 aldehyde, C12-C16 acetate ester, or C12-C16 primary alcohol, a housing, and one or more dispensers (e.g., one or more pheromone dispensers for dispensing one or more of the active components). Thus, in some embodiments, the presently disclosed subject matter provides a multi-component device for attracting and capturing a male agricultural pest of a Spodoptera species, the device comprising: (a) a first active component comprising at least one C7-C11 aldehyde; (b) a second active component comprising at least one C12-C16 aldehyde, acetate ester, or primary alcohol; (c) a housing comprising one or more opening for entry of said male agricultural pest; and (d) one or more dispensers, wherein first active component (a) is incorporated into at least one of said one or more dispensers and wherein second active component (b) is incorporated into at least one of said one or more dispensers. In some embodiments, at least one of said one or more dispensers is made of a chemically neutral material selected from the group comprising a polymer, a glass, a rubber, an elastomer, cellulose, wood, and felt.

In some embodiments, the housing further comprises a mount (e.g., a bracket and/or fastening mechanism) or hanger configured to mount or hang the device in a fixed location. In some embodiments, the housing further comprises an insert comprising an adhesive that can adhere to said pest to keep said pest from exiting the housing.

In some embodiments, the device further comprises (e) a further active agent. In some embodiments, the further active agent comprises a killing agent, a slow acting insecticide, or a biological agent. In some embodiments, the biological agent is selected from the group comprising a bacteria, a virus, a fungi, and a nematode.

### III. Methods

In some embodiments, the presently disclosed subject matter relates to the use of nonanal (e.g., compositions comprising nonanal) for attracting FAW (Spodoptera frugiperda) and/or other Spodoptera species. In some embodiments, at least one, two, three, four or more previously identified FAW sex pheromone components (e.g., at least one, two, three, four or more C12-C16 acetate esters and/or aldehydes and/or alcohols (e.g., primary alcohols), such as at least one, two, three, four or more C12-, C14-, or C16 acetate esters and/or aldehydes and/or alcohols (e.g., primary alcohols)) can be used in combination with nonanal. In addition to, or as an alternative to nonanal similar compounds, such as heptanal (7:Ald), octanal (8:Ald), decanal (10:Ald) and undecanal (11:Ald) can be used. Although to date these other C7-C11 aldehyde compounds have not been found in the FAW female's pheromone gland, it is possible that FAW pheromone receptors can be broadly tuned to nonanal-related aldehydes and/or that one or more of these other aldehydes will be detected in more concentrated FAW female pheromone gland extracts.

In some embodiments, the presently disclosed subject matter provides a method of attracting a male agricultural pest of the Spodoptera species, such as a male Spodoptera frugiperda, wherein the method comprises providing one or more baits or lures collectively comprising (a) a first active component comprising a C7-C11 aldehyde and (b) a second active component comprising one or more saturated or unsaturated C12-C16 aldehyde, C12-C16 acetate ester or C12-C16 primary alcohol. In some embodiments, the method further comprises estimating a Spodoptera species population size and/or controlling the size of such a population (e.g., by disrupting mating of Spodoptera or by temporarily or permanently removing male Spodoptera from the population).

In some embodiments, the agricultural pest is a Spodoptera frugiperda (FAW). In some embodiments, the agricultural pest further comprises one or more additional Spodoptera species as described hereinabove. In some embodiments, the agricultural pest is one or more Spodoptera species other than Spodoptera frugiperda.

In some embodiments, the first active component comprises, consists essentially of, or consists of nonanal. In some embodiments, the second active component comprises, consists essentially of, or consists of a C12 acetate ester and/or a C14 acetate ester and/or C16 acetate ester. In some embodiments, said C12 acetate ester and/or said C14 acetate ester and/or said C16 acetate ester comprises one or more alkene group. In some embodiments, said C12 acetate ester and/or said C14 acetate ester and/or said C16 acetate ester comprises one alkene group (i.e., comprises a monounsaturated carbon chain). In some embodiments, said second active component comprises one or more of the group comprising Z9-14:OAc, Z7-12:OAc, Z9-12:OAc, and Z11-16:OAc. In some embodiments, the second active component comprises at least two of Z9-14:OAc, Z7-12:OAc, Z9-12:OAc, and Z11-16:OAc. In some embodiments, the second active component further comprises one or more additional components (i.e., one or more additional C12-C16 aldehyde, acetate ester or primary alcohol). In some embodiments, the one or more additional components are selected from the group comprising Z10-14:OAc, 14:OAc, Z11-14:OAc, E7-12:OAc, 12:OAc, Z11-12:OAc, Z9-14:Ald, and Z11-16:Ald.

In some embodiments, the first active component comprises, consists essentially of, or consists of nonanal and the second active component comprises Z9-14:OAc. In some embodiments, the nonanal is present in an amount ranging from about 0.10 wt % to about 50 wt % (e.g., about 0.10, 0.50, 1.0, 5.0, 10, 15, 20, 25, 30, 35, 40, 45, or about 50 wt %) compared to the weight of the Z9-14:OAc. In some embodiments, the nonanal is present in an amount ranging from about 0.10 wt % to about 10 wt % (e.g., about 0.10, 0.20, 0.30, 0.40, 0.50, 0.60, 0.70, 0.80, 0.90, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or about 10 wt %) compared to the weight of the Z9-14:OAc. In some embodiments, the nonanal is present at about 0.50 wt % to about 5 wt % compared to the weight of the Z9-14:OAc. In some embodiments, the nonanal is present at about 1 weight % compared to the weight of the Z9-14:OAc.

The first and second active components can be formulated separately or together. In some embodiments, the first and second active components are formulated separately. In some embodiments, the first active component is provided in a separate dispenser from the second active component. Thus, in some embodiments, the first active component is provided in a first dispenser and the second active component is provided in a second dispenser. In some embodiments, one or both of the first and the second active component is formulated in a slow release formulation. In some embodiments, the slow release formulation comprises an oil, such as paraffin oil or another odorless, non-volatile oil. In some embodiments, the first active component is formulated in an oil and provided in a first dispenser and the second active component is provided in a second dispenser. In some embodiments, the second dispenser comprises a chemically neutral material selected from the group comprising a polymer, a glass, a rubber, an elastomer, cellulose, wood, and felt. In some embodiments, the second dispenser comprises rubber.

In some embodiments, the one or more baits or lures are provided in association with a housing for trapping one or more pest and the method further comprises collecting one or more male agricultural pest of a Spodoptera species (e.g., male FAW) in the housing. In some embodiments, the method further comprises estimating a pest population size based upon analyzing the number of pests trapped in the housing. In some embodiments, the method further comprises keeping trapped male pests in said housing or transferring said trapped pests to another housing, thereby controlling a pest population by removing male pests from the total pest population and reducing the number of male pests available for mating.

In some embodiments, the method further comprises controlling a pest population by treating attracted (or attracted and trapped) male pests (e.g., male FAW) with a slow-acting insecticide or biological control agent; and releasing the treated male pests, wherein the treated male pests transfer the slow-acting insecticide or biological control agent to female pests upon mating. In some embodiments, the method further comprises controlling a pest population by treating the attracted (or attracted and trapped) male pests with a pest killing agent. In some embodiments, the pest killing agent is a fast-acting insecticide.

In some embodiments, the method further comprises controlling a pest population by providing a plurality of the one or more baits or lures to a select location, thereby inundating the location with the first and second components to confuse male agricultural pests and make it more difficult for said male agricultural pests to locate a mate.

In some embodiments, the method is performed at or near a port of entrance, e.g., to estimate the population of a pest species at or near said port of entrance associated with goods being imported or exported from a country. For instance, the method can be performed at or near an imported container at a harbor, airport, roadway and/or train border crossing, to detect the presence or absence of said pest.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

### General Materials and Methods

Electrophysiology: The sex pheromone gland of ten 3-4-day old FAW females was dissected and placed in a glass insert containing 30 μl hexane (SupraSolv®; MilliporeSigma, Burlington, Mass., United States of America). After 10 min, the hexane extract was transferred to a second insert and concentrated down to 10 μl under a gentle flow of nitrogen.

A gas chromatograph coupled to an electroantennogram detector (GC-EAD) was used to identify biologically active compounds that are perceived by the antenna of FAW males, since the antenna is the main olfactory organ of insects. The GC separates chemical mixes into individual compounds and directs them to the insect antenna, which is connected to an amplifier, the EAD. In parallel to the GC, the electrophysiological response of the antenna is recorded to identify the active compounds. An aliquot of 1 μl of the pheromone gland extract was injected in the GC-EAD where a male FAW antenna was mounted. Clean hexane that was handled in the same manner without glands was used as control.

Identification of the new EAD active compound: Gland extracts were analyzed on a GC-MS (6890 GC and 5975 MS, Agilent Technologies, Palo Alto, Calif., United States of America) and operated in pulsed splitless mode (15 psi for 0.5 min, then 6 psi) and equipped with a DB-WAXetr column (30 m×0.25 mm, df=0.25 μm, Agilent Technologies, Palo Alto, Calif., United States of America), and helium was used as the carrier gas at an average velocity of 34 cm/s. The oven program was set to 40° C. for 2 min, increased at 10° C./min to 250° C. Injector temperature was set to 250° C., transfer line temperature was 260° C., and MS quadrupole was 150° C. The mass-to-charge ratio range was from 33 to 650. Compounds were identified based on Kovats indices, electron ionization mass spectra and comparison with authentic synthetic standards.

Preparation of synthetic mix: Synthetic compounds were purchased from commercial vendors. Z9-14:OAc was purchased from Bedoukian Research Inc. (Danbury, Conn., United States of America). Z7-12:OAc was a gift from Prof. Kenneth Haynes from the University of Kentucky, but can also be purchased from commercial vendors, such as Bedoukian Research Inc, (Danbury, Conn., United States of America) and Millipore Sigma (Burlington, Mass., United. States of America). These compounds were diluted in hexane to produce stock solutions of 10 and 0.1 μg/μL, respectively, Nonanal was purchased from Sigma-Aldrich (St. Louis, Mo., United States of America). For behavioral assays in laboratory conditions (olfactometer), nonanal was diluted in hexane to produce a stock solution of 0.1 μg/μL. For field assays, nonanal was diluted in paraffin oil (ICN Biomedicals, Costa Mesa, Calif., United States of America) at different concentrations (see Field Assays).

Behavioral Assay: A no-choice linear olfactometer assay was used to test the behavior of FAW males to different formulations. The olfactometer consists of a linear Plexiglas® tube (n=6 cm i.d., length=145 cm) that was connected to an air pump that generated an internal airflow speed of 0.1 m/s. A short Plexiglas® chamber filled with activated charcoal was connected to the opposite end of the olfactometer to filter the inward air. A virgin male moth (3-4 days old) that did not have any contact with female moths was placed inside a release cage that was mounted downwind, before the air pump Formulations were prepared in hexane. An aliquot of 10 was added to a piece of filter paper and solvent was left to evaporate for 30 min at room temperature. Filter papers were introduced to the olfactometer through an opening (0=2 cm) on the top of the olfactometer, 4 cm away from the charcoal filter. Filter papers were tested only once. Moths were tested for 5 minutes and the sequence of their behavioral response was recorded as: activation (wing fanning and start moving upwind), reaching the half-way point of the olfactometer (HW), close approach to the odor source (CA), and source contact (contact). The experiment was conducted 4-7 hours into the scotophase.

The tested formulations were:

Formulation #1

| | | |
|---|---|---|
| Pheromone compound | Amount | Percentage |

| | | | |
|---|---|---|---|
| Z9-14:OAc | 100 | ng | 99.42% |
| Z7-12:OAc | 0.58 | ng | 0.58% |
| Nonanal | 0 | ng | 0% |

Formulation #2

| | | |
|---|---|---|
| Pheromone compound | Amount | Percentage |

| | | | |
|---|---|---|---|
| Z9-14:OAc | 100 | ng | 99.37% |
| Z7-12:OAc | 0.58 | ng | 0.58% |
| Nonanal | 0.05 | ng | 0.05% |

Formulation #3

| | | |
|---|---|---|
| Pheromone compound | Amount | Percentage |

| | | | |
|---|---|---|---|
| Z9-14:OAc | 100 | ng | 99.32% |
| Z7-12:OAc | 0.58 | ng | 0.58% |
| Nonanal | 0.1 | ng | 0.1% |

Formulation #4

| | | |
|---|---|---|
| Pheromone compound | Amount | Percentage |

| | | | |
|---|---|---|---|
| Z9-14:OAc | 100 | ng | 98.434% |
| Z7-12:OAc | 0.58 | ng | 0.57% |
| Nonanal | 1 | ng | 1% |

Field Assays: Formulations were tested under field conditions. Z9-14:OAc and Z7-12:OAc were diluted in hexane and 100 μL of the solution containing 100 μg and 0.58 μg of the respective compounds was loaded to a rubber septum (11 mm; Wheaton, DWK Life Sciences, Millville, N.J., United States of America). The septum was left inside a fume hood for 15 hr for the solvent to evaporate and then tested in field assays. Nonanal was diluted in paraffin oil and 100 μL of the solution was transferred to a vial dispenser. The solution contained either 0.1 μg or 1 μg, which corresponded to 0.1% and 1% of the amount of Z9-14:OAc. This dispenser constituted a 2 ml borosilicate vial covered with aluminum foil and contained 50 mg of deactivated glass wool. The vial was loaded with a nonanal solution and capped. The autosampler cap had a silicone septum with a PTFE liner through which a microcapillary glass was inserted (L: 1.25 inch; O.D.: 0.034 inch; I.D.: 0.0157 inch; 4 μL internal volume) to allow the volatile nonanal to leave the vial. A straightened metal paper clip pierced through the small cup of a pheromone-loaded rubber septum and was tied around the neck of a nonanal-loaded vial. This set was held by a steel alligator clip installed inside a Hartstack trap. We tested the attraction of male FAW to the following treatments:
Control: clean hexane loaded into a rubber septum and clean paraffin oil in a, vial dispenser
Pheromone mix: 100 μj of Z9-14:OAc and 0.58 μg of Z7-12:OAc in rubber septum and clean paraffin oil in vial dispenser
Pheromone 4-0.1% Nonanal: Pheromone mix in rubber septum and 0.1 μg of nonanal in vial dispenser
Pheromone+1% Nonanal: Pheromone mix in rubber septum and 1 μg of nonanal in vial dispenser

The Hartstack trap consisted of a conical body made of metal mesh with a removal top (H: 15 cm, ø=14 cm) in which males were trapped. Traps stood over the canopy of crops on rebars (L: 1.5 m) that were fixed to the ground. Traps were set for one week in a cotton field along the crop row, 15 m apart. Each experimental block consisted of 4 traps 15 m from each other. The next block was set 15 m from the first block and parallel to it. Each treatment was replicated once within a block and assigned randomly to trap positions. The number of trapped male FAW moths and other species were counted daily and traps were rotated.

A second study repeated the same procedure described above but increased the amount of Z9-14:OAc and Z7-12:OAc in the mix to 1000 μg and 5.8 μg, respectively, and nonanal to 10, 20 and 40 μg. The attraction of male FAW was tested to the following treatments:
Control: clean hexane loaded into a rubber septum and clean paraffin oil loaded into a vial dispenser
Pheromone mix: 1000 μg of Z9-14:OAc and 5.8 μg of Z7-12:OAc in rubber septum and clean paraffin oil in vial dispenser (no nonanal).
Pheromone 4-1% Nonanal: Pheromone mix in rubber septum and 10 μg of nonanal in vial dispenser.
Pheromone+2% Nonanal: Pheromone mix in rubber septum and 20 μg of nonanal in vial dispenser.
Pheromone+4% Nonanal: Pheromone mix in rubber septum and 40 μg of nonanal in vial dispenser.

Field test with commercial formulation: The effect of adding nonanal to commercial formulations was tested in a sorghum crop for five days. Lures were purchased from two commercial suppliers, referred to herein as “Commercial Supplier A” (L105A from Seemly Biologicals Inc., Billings, Mont., United States of America) and “Commercial Supplier B” (FAW 3143 from Trécé, Adair, Okla., United States of America). These lures were formulated by the respective companies in rubber septa. Nonanal was formulated in the same way as described above and a straightened metal paper clip held together both the nonanal-loaded vial and the commercial lure. This set was installed inside a Hartstack trap and traps were positioned in the same manner described above. The attraction of male FAW to the following treatments was tested:
Commercial Supplier B lure combined with clean paraffin oil in vial dispenser (no nonanal).
Commercial. Supplier B lure combined with 20 μg of nonanal in vial dispenser.
Commercial Supplier A lure combined with clean paraffin oil in vial dispenser (no nonanal).
Commercial Supplier A lure combined with 20 μg of nonanal in vial dispenser.

A second study was conducted in the same crop over a second five-day period, aimed to investigate different doses of nominal in combination with Commercial Supplier A lures. Using the same procedure described above, the attraction of male FAW was tested to the following treatments:
Commercial. Supplier A lure combined with clean paraffin oil in vial dispenser (no nonanal).
Commercial Supplier A lure combined with 10 μg of nonanal in vial dispenser.
Commercial Supplier A lure combined with 20 μg of nonanal in vial dispenser.
Commercial Supplier A lure combined with 40 μg of nonanal in vial dispenser.
Commercial Supplier A lure combined with 80 μg of nonanal in vial dispenser.

Statistical analysis: All statistical analyses were performed in R (version 3.5.1). Behavioral responses of male FAW to formulations in the olfactometer were analyzed by binomial generalized linear model. Numbers of male FAW catches in the same day were combined per treatment and analyzed by Poisson generalized linear model (GLM). Treatments that had no catches were not included in the analyses.

### Example 2

### Electrophysiology and Chemical Analysis

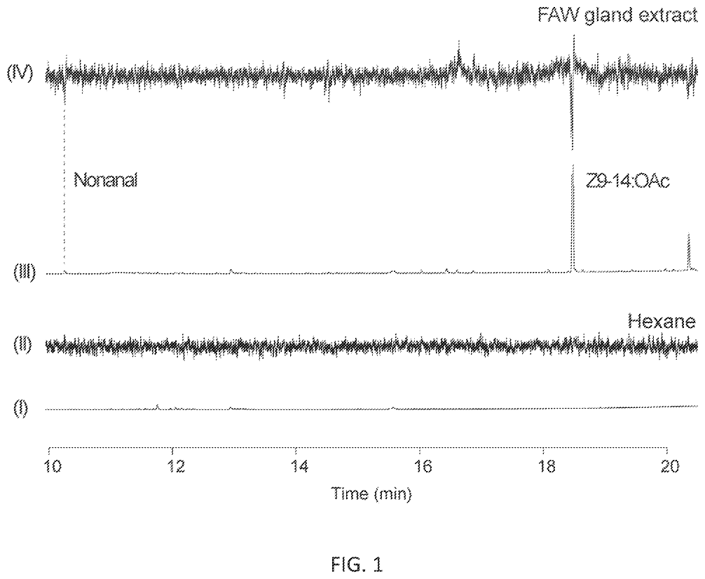

Active pheromone gland compounds were recognized using gas chromatography coupled to electroantennographic detection (GC-EAD) with male antennae. No compound in the clean hexane (control) elicited any FAD response. See FIGS. 1, I and II. In addition to known pheromone components, one extra active compound from the gland extract eluted at a much earlier retention time (10.2 min; see FIG. 1, III and IV) and was identified by mass spectrometry as nonanal. The amount of nonanal was 1.12% relative to the main pheromone component, Z9-14:OAc.

### Example 3

### Behavioral Response

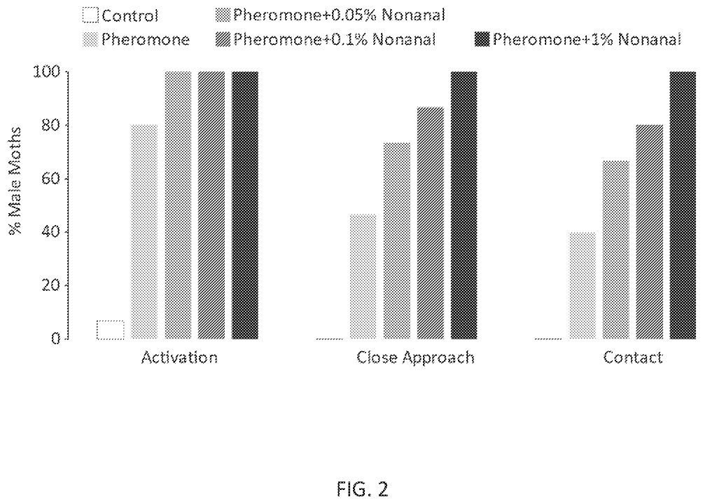

The addition of nonanal to a synthetic 2-component pheromone formulation enhanced the attraction of male FAW in an olfactometer assay. See FIG. 2. The 2-component pheromone formulation with Z9-14:OAc and Z7-12:OAc activated 80% of tested males but only 40% reached the pheromone source. However, the addition of nonanal at all doses increased the activation of males to 100% and more than 66% reached the source. Among these, the addition of 1% nonanal yielded the most attractive formulation and induced 100% of tested males to reach the pheromone source. The control hexane only activated 6.6% of males none of which reached the pheromone source.

### Example 4

### Field Tests

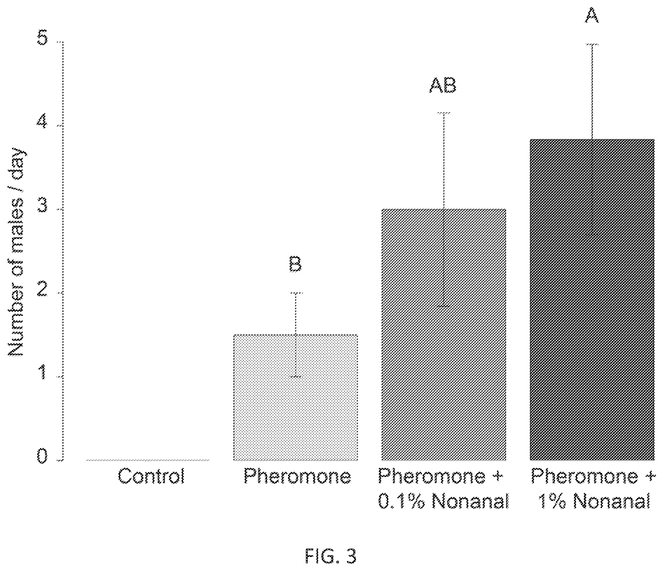

The effect of nominal when added to a 2-component pheromone formulation (100 μg of Z9-14:OAc and 0.58 μg of Z7-12:OAc) on field catches of male FAW in a cotton field. Nonanal was tested at different doses, 0.1 and 1 μg, which correspond to 0.1% and 1% of the amount of the main pheromone component, Z9-14:OAc. The addition of nonanal significantly increased the numbers of male FAW in traps with the addition of 1% nonanal improving the catches by 155% compared to the pheromone mix alone. See FIG. 3. The addition of 0.1% nonanal had an intermediate trap catch between the pheromone mix alone and with the addition of 1% nonanal. Control traps did not catch any males.

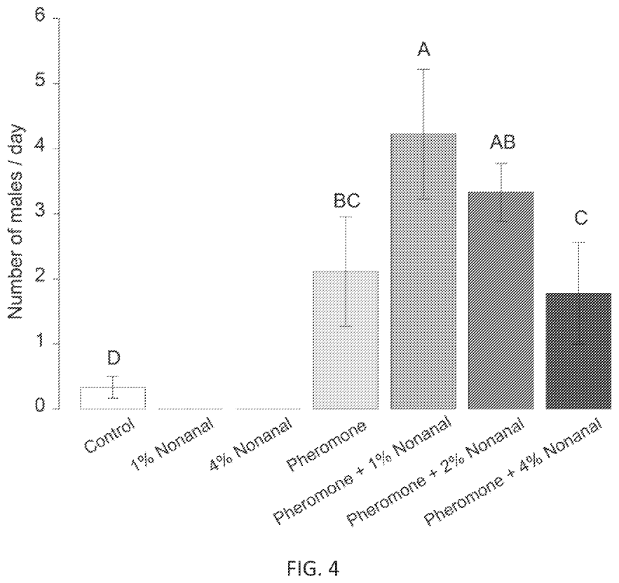

In a second assay at the same plot, the amount of the pheromone components Z9-14:OAc and Z7-12:OAc was increased to 1 mg and 5 μg, respectively, and included higher proportions of nonanal. As in the previous experiment, adding 1% nonanal to the pheromone formulation yielded the highest number of male catches per day (100% more than the catches by the pheromone alone), while the addition of 2% and 4% nonanal decreased the number of males caught and was not significantly different from the pheromone formulation alone. See FIG. 4. Nonanal alone did not yield any catch. See FIG. 4.

### Example 5

### Field Tests with Modified Commercial Lures

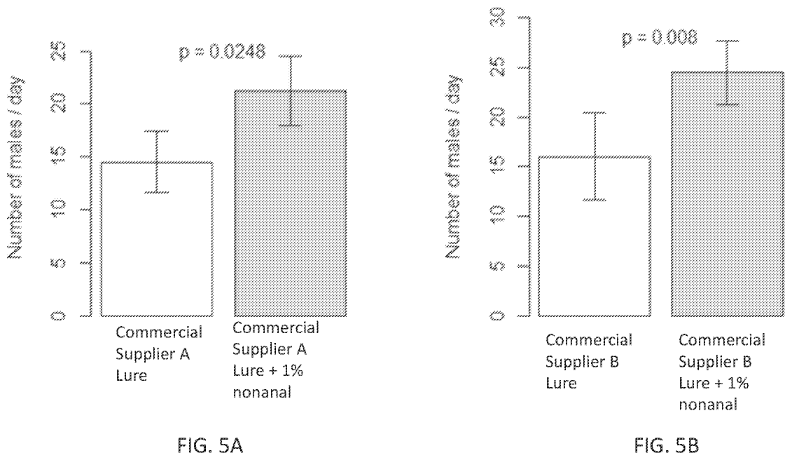

Nonanal was added to commercial lures from two companies, Commercial Supplier A (Scenery Biologicals, Billings, Mont., United States of America) and Commercial Supplier B (Trece, Adair, Okla., United States of America). Unlike the formulations used in Example 4, which contained 2 components, these lures contained 4 components, namely Z9-14:OAc, Z7-12:OAc, Z9-12:OAc and Z11-16:OAc. Based on communication with company representatives, the amount of the main pheromone component, Z9-14:OAc, in commercial lures was estimated to be 2 mg. Since the addition of 1% nonanal relative to the main pheromone component yielded the highest trap catches in the field tests in Example 4, 20 μg (1%) nonanal was added to these commercial lures. The addition of nonanal significantly increased the number of males caught per day compared to the commercial lures alone but not as high as the number of males caught in the field tests of Example 4. See FIGS. 5A and 5B. Nonanal increased the trap catches by 46.5% and 53% for Commercial Supplier A and Commercial Supplier B lures, respectively.

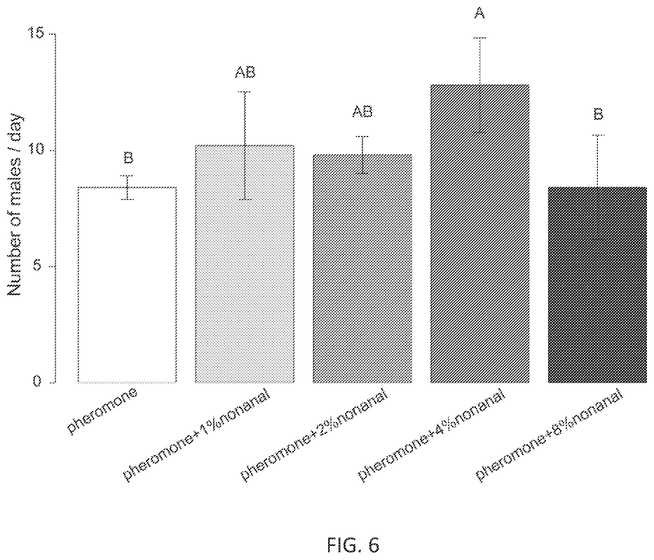

Since the addition of 20 μg nonanal (1%) to commercial lures did not double the number of males caught, as previously observed with addition to the two-component pheromone formulation, the effect of other nonanal doses was investigated using Commercial Supplier A lures. Results show that increasing the amount of nonanal to 80 μg (4%) increased the number of males caught to 52.4% compared to the commercial lure alone. See FIG. 6. The addition of 1% and 2% only slightly increased the trap catches while the addition of 8% decreased trap catch, See FIG. 6.

In summary, a new FAW pheromone component that was never described before and that significantly increases the number of male moths caught in field assays was identified. Nonanal is a short-chain aldehyde commonly detected in plants and some animals, like birds, but not in moths. Previous studies have only evaluated the FAW sex pheromone gland using chemical techniques and behavioral assays but without any electrophysiological data from the antenna. The simple addition of nonanal at minute amounts can improve surveillance, monitoring and control tools for this pest that has globally spread.

Accordingly, the addition of nonanal to prior FAW pheromone lures can improve any application that requires attracting males of FAW to traps, insecticides, or other pest management devised. These applications include the detection of pest, including surveillance systems to detect FAW where it is not been previously present, such as in ports of entrance, including in containers in harbors or airports, as well as in monitoring systems used to estimate the population size of RAW. Monitoring is a step of integrated pest management that informs farmers whether a control method needs to be applied before economic loss, it can also inform farmers if pest control efforts have been successful (i.e., killed larvae and adult moths, so that fewer moths are trapped). Applications for the control of pests include trap-and-kill methods to reduce the FAW population in agricultural fields that comprise attracting male moths to a dispenser that also contains a killing agent (e.g., an insecticide). Other control methods include mass trapping, which involves attracting large numbers of moths to traps that contain the pheromone, thus removing the males from the population. Another control method is mating disruption, which comprises inundating a field with a pheromone, e.g., by applying dispensers of pheromone in multiple locations. Male moths are then confused by the synthetic pheromones and have difficulty locating females that are releasing pheromones, ultimately disrupting the FAW life cycle. Autoinoculation is a control method where males are attracted to a pheromone lure, where they are dosed with a slow-acting insecticide or biological control agent (e.g., bacteria, viruses, fungi, nematodes, etc.), which they then transfer to females upon mating.

In addition to the Commercial Supplier A and Commercial Supplier B lures described herein, nonanal can also be added to other commercial lures for FAW (e.g., including two component lures containing Z9-14:OAc and Z7-12:OAc; four component lures containing Z9-14:OAc, Z7-12:OAc, Z9-12:OAc, and Z11-16:OAc, or other formulations containing any of the previously identified FAW pheromone components). Other aldehydes, such as heptanal, octanal, decanal, and undecanal, which are structurally similar to nonanal but with different hydrocarbon chain lengths, could also be included to improve current commercial lures, either in combination with or separate from nonanal.

It will be understood that various details of the presently disclosed subject matter can be changed without departing from the scope of the presently disclosed subject matter. Furthermore, the foregoing description is for the purpose of illustration only, and not for the purpose of limitation.

1. A composition for attracting a male agricultural pest of a Spodoptera species, wherein said composition comprises:
(a) a first active component comprising at least one C7-C11 aldehyde; and
(b) a second active component comprising at least one C12-C16 aldehyde, acetate ester, or primary alcohol.

2. The composition of claim 1, wherein the agricultural pest is Spodoptera frugiperda.

3. The composition of claim 1, wherein the first active component comprises or consists of nonanal.

4. The composition of claim 1, wherein the second active component comprises a C12 acetate ester and/or a C14 acetate ester and/or a C16 acetate ester, optionally wherein said C12 acetate ester and/or C14 acetate ester and/or C16 acetate ester comprises an alkene group.

5. The composition of claim 4, wherein the second active component comprises (Z)-9-tetradecenyl acetate (Z9-14:OAc), (Z)-7-dodecenyl acetate (Z7-12:OAc), (Z)-9-dodecenyl acetate ester (Z9-12:OAc), (Z)-11-hexadecenyl acetate (Z11-16:OAc) or a combination thereof; optionally wherein the second active component comprises at least two of Z9-14:OAc, Z7-12:OAc, Z9-12:OAc, and Z11-16:OAc; further optionally wherein the second active component further comprises one or more additional component selected from the group consisting of (Z)-10-tetradecenyl acetate (Z10-14:OAc), tetradecyl acetate (14:OAc), (Z)-11-tetradecenyl acetate (Z11-14:OAc), (E)-7-dodecenyl acetate (E7-12:OAc), dodecyl acetate (12:OAc), (Z)-11-dodecenyl acetate (Z11-12:OAc), (Z)-9-tetradecenal (Z9-14:Ald), and (Z)-11-hexadecenal (Z11-16:Ald).

6. The composition of claim 1, wherein one or both of the first and the second active component is formulated in a slow release formulation, optionally wherein said first active component is formulated in an oil.

7. The composition of claim 1, wherein the first active component comprises nonanal and the second active component comprises Z9-14:OAc; and wherein the nonanal is present at an amount ranging from about 0.10 weight % to about 50 weight % compared to the weight of the Z9-14:OAc.

8. The composition of claim 7, wherein the nonanal is present at about 1 weight % compared to the weight of the Z9-14:OAc.

9. The composition of claim 1, wherein the first active component and the second active component are separately formulated.

10. The composition of claim 1, further comprising a pest killing agent, a slow-acting insecticide, or a biological agent, optionally wherein said biological agent is selected from a bacteria, a fungi, a virus, and a nematode, optionally wherein said pest killing agent is a fast-acting insecticide.

11. A method of attracting a male agricultural pest of a Spodoptera species, the method comprising providing one or more baits or lures, wherein said one or more baits or lures collectively comprise (a) a first active component comprising at least one C7-C11 aldehyde; and (b) a second active component comprising at least one C12-C16 aldehyde, acetate ester, or primary alcohol.

12. The method of claim 11, wherein the agricultural pest is Spodoptera frugiperda.

13. The method of claim 11, wherein the first active component comprises or consists of nonanal.

14. The method of claim 11, wherein the second active component comprises a C12 acetate ester and/or a C14 acetate ester and/or C16 acetate ester, optionally wherein said C12 acetate ester and/or said C14 acetate ester and/or said C16 acetate ester comprises an alkene group.

15. The method of claim 14, wherein the second active component comprises (Z)-9-tetradecenyl acetate (Z9-14:OAc), (Z)-7-dodecenyl acetate (Z7-12:OAc), (Z)-9-dodecenyl acetate ester (Z9-12:OAc), (Z)-11-hexadecenyl acetate (Z11-16:OAc) or a combination thereof; optionally wherein the second active component comprises at least two of Z9-14:OAc, Z7-12:OAc, Z9-12:OAc, and Z11-16:OAc; further optionally wherein the second active component further comprises one or more additional component selected from the group consisting of (Z)-10-tetradecenyl acetate (Z10-14:OAc), tetradecyl acetate (14:OAc), (Z)-11-tetradecenyl acetate (Z11-14:OAc), (E)-7-dodecenyl acetate (E7-12:OAc), dodecyl acetate (12:OAc), (Z)-11-dodecenyl acetate (Z11-12:OAc), (Z)-9-tetradecenal (Z9-14:Ald), and (Z)-11-hexadecenal (Z11-16:Ald).

16. The method of claim 11, wherein one or both of the first and the second active component is formulated in a slow release formulation, optionally wherein the first component is formulated in an oil.

17. The method of claim 11, wherein the first active component comprises nonanal and the second active component comprises Z9-14:OAc; and wherein the nonanal is present in an amount ranging from about 0.10 weight % to about 50 weight % compared to the weight of the Z9-14:OAc.

18. The method of claim 17, wherein the nonanal is present at about 1 weight % compared to the weight of the Z9-14:OAc.

19. The method of claim 11, wherein the first active component and the second active component are separately formulated, optionally wherein the first active component is provided in a separate dispenser from the second active component.

20. The method of claim 19, wherein the first active component is formulated in an oil and provided in a first dispenser and the second active component is provided in a second dispenser, optionally wherein said second dispenser comprises rubber.

21. The method of claim 11, wherein the one or more baits or lures are provided in association with a housing for trapping one or more pest and the method further comprises collecting one or more male agricultural pest of a Spodoptera species in the housing.

22. The method of claim 21, further comprising estimating a pest population size based upon analyzing the number of pests trapped in the housing.

23. The method of claim 21, wherein the method further comprises keeping trapped male pests in said housing or transferring said trapped pests to another housing, thereby controlling a pest population by removing male pests from the total pest population and reducing the number of male pests available for mating.

24. The method of claim 11, wherein the method further comprises controlling a pest population by treating attracted, optionally trapped, male pests with a slow-acting insecticide or biological control agent; and releasing the treated male pests, wherein the treated male pests transfer the slow-acting insecticide or biological control agent to female pests upon mating.

25. The method of claim 11, wherein the method further comprises controlling a pest population by providing a plurality of the one or more baits or lures to a select location, thereby inundating the location with the first and second components to confuse male agricultural pests and make it more difficult for said male agricultural pests to locate a mate.

26. The method of claim 11, wherein the method further comprises controlling a pest population by treating the attracted, optionally trapped male pests with a pest killing agent, optionally a fast-acting insecticide.

27. The method of claim 11, wherein the method is performed at or near a port of entrance, optionally at or near an imported container at a harbor, airport, roadway and/or train border crossing, to detect the presence or absence of said pest.

28. A multi-component device for attracting and capturing a male agricultural pest of a Spodoptera species, the device comprising:
(a) a first active component comprising at least one C7-C11 aldehyde;
(b) a second active component comprising at least one C12-C16 aldehyde, acetate ester, or primary alcohol;
(c) a housing comprising one or more opening for entry of said male agricultural pest, optionally wherein said housing further comprises a mount configured to mount the device in a fixed position; and
(d) one or more dispensers, wherein first active component (a) is incorporated into at least one of said one or more dispensers and wherein second active component (b) is incorporated into at least one of said one or more dispensers.

29. The device of claim 28, wherein at least one of said one or more dispensers is made of a chemically neutral material selected from the group consisting of a polymer, a glass, a rubber, an elastomer, cellulose, wood, and felt.

30. The device of claim 28, wherein the housing further comprises an insert comprising an adhesive that can adhere to said pest to keep said pest from exiting the housing.

31. The device of claim 28, further comprising (e) a further active agent comprising a killing agent, a slow acting insecticide, or a biological agent, optionally wherein said biological agent is selected from the group consisting of a bacteria, a virus, a fungi, and a nematode.
